(19) United States
(12) Patent Application Publication
(10) Pub. No.: US 2024/0382281 A1
(43) Pub. Date: Nov. 21, 2024

(54) IMAGING-GUIDED WHOLE-BODY STEREOTACTIC DEVICE

(71) Applicant: Ashish Mitra, Sydney NSW (AU)

(72) Inventor: Ashish Mitra, Sydney NSW (AU)

(21) Appl. No.: 18/684,662

(22) PCT Filed: Aug. 20, 2022
(86) PCT No.: PCT/AU2022/050933

§ 371 Date: Feb. 17, 2024

(30) Foreign Application Priority Data
Aug. 20, 2021 (AU) 2021902614

(51) Int. Cl. A61B 90/11; A61B 90/00

(52) U.S. Cl. CPC A61B 90/11; A61B 90/06; A61B 90/37; A61B 2090/067; A61B 2090/374; A61B 2090/3762

(57) ABSTRACT

Presented is an imaging (including cross-sectional imaging) guided whole-body stereotactic device adaptable to align and precisely orient a variety of medical devices/instruments such as differently sized needles, electrodes etc. into a patient's body for carrying out intended medical procedures in a minimally invasive manner. The proposed device is an assembly of multiple parts accompanied by a software program product.

### CROSS-REFERENCE TO RELATED PATENT DOCUMENTS

This patent application claims the benefit of priority of Australian provisional Patent Application No. 2021902614, titled “IMAGING-GUIDED WHOLE-BODY STEREOTACTIC DEVICE”, filed on Aug. 20, 2021, which is hereby incorporated by reference in its entirety.

### TECHNICAL FIELD OF INVENTION

This invention relates generally to medical devices. The present invention in general relates to stereotactic devices. More particularly, the present invention relates to an imaging-guided whole body stereotactic device adaptable to align and precisely orient a variety of medical devices/instruments such as differently sized needles, electrodes etc. into a patient's body for carrying out intended medical procedures in the patient such as carrying out tumor biopsies, draining of brain abscess, carrying out radiofrequency ablation etc.

### BACKGROUND

In medical field, it is often necessary to precisely orient and guide a variety of medical devices/instruments such as differently sized needles, electrodes etc. at a particular part of the body or an organ deep inside the body. This may be required for obtaining tissue samples for the purpose of diagnosis, for delivering drugs/energy, for therapeutic/palliative aspiration of fluid collections or for procedures such as tumor biopsies, percutaneous discectomies, cyst aspirations, tumor localizations and such.

Such procedures are usually carried out percutaneously under image guidance obtained from cross sectional imaging devices such as ultrasound/CT scan/MRI scan etc. Image guidance is required to select least harmful path for guiding the instruments within the body, so as to avoid damages to vital organs and structures such as blood vessels, bowel etc. Although it is possible to determine exact location using such CT scan/MRI scan techniques, guiding the instruments, such as needle, to a desired precise location using a free hand involves trial and error and often requires multiple attempts. At times, despite multiple attempts it may not be possible to place the instrument at the desired precise location in the body. At times, multiple attempts of passing the medical device within the body may cause serious life-threatening complications of internal bleeding and/or damage to vital organs that may fall in the path of the instrument (such as needle). Additionally, lifesaving operations/procedures require precise placement of needle/medical devices in the body avoiding damage to other delicate organs, tissues, blood vessels etc. Often while performing the procedure free hand, it is not possible to place the needle in the precise location the first time. Often there is a resultant situation of inserting the instrument (such as needle) multiple times in order to the needle/medical devices in the precise location and this may lead to serious clinical complications.

The act of orienting and guiding the medical devices such as needles can be better performed using some guiding devices (herein after referred to as ‘stereotactic devices’) that can guide the instruments such as a needle in the precise direction to reach the precise point/location in the body in the first attempt itself.

In the past, several inventors have proposed different kinds of stereotactic devices that may be used to precisely position needles or similar medical devices. For example, U.S. Pat. No. 4,733,661 discloses a hand-held needle guidance device suitable to accurately and easily use CT generated information to position a biopsy needle or drainage catheter relative to a patient's body. There also exists a highly complex CT scanner guided stereotactic brain surgery device that utilizes skull mounted frames with associated complex positioning instruments.

U.S. Pat. No. 4,583,538 discloses an apparatus designed to facilitate CT guided biopsies of the body. The apparatus disclosed in this patent is floor-mounted and is designed to position a needle guide by moving the same through any of three perpendicular axes. Angular rotations about such axes are permitted to orient the needle guide in any desired direction.

US20060009787 discloses a device that includes a frame, with puncture guides for guiding the tip of a puncturing needle to a predetermined position within the brain, and right and left fixing frames respectively having fixing needles for fixing the device on the patient head, the fixing frames being displaceable in a longitudinal direction of the frame, and the frame being provided with a plurality of guides for guiding the tip of a puncturing needle toward a point on a line connecting the right and left fixing needles.

The devices available for guiding the needle for brain interventions are not suitable/compatible for use in other parts of the body. Further, the devices discussed herein above are either complex or limited in their use for some specific locations of the body and thus cannot be universally used for guiding the medical devices such as needles to any desired location or point within any part of the body.

None of the existing stereotactic devices are capable of being used satisfactorily in body parts affected by respiratory movement. In order to prevent damage to body organs and tissues in the path of the guided medical devices or instruments such as needles during respiratory movements it is necessary to allow free movement of the needle during breathing. The present invention provides a stereotactic device that obviates the above limitations. The main object of the present invention is to provide an imaging (including cross-sectional imaging) guided stereotactic device for needle/medical device placement that could be used for interventions in the entire of the body including brain, thorax, abdomen, limbs and such.

### SUMMARY

Embodiments of the present invention discloses a computerized tomography (CT) and Magnetic Resonance Imaging (MRI) compatible imaging-guided stereotactic device useful for inserting differently sized needles or any such apparatus/medical devices/instruments at a desired precise location within the body.

It is an object of the present invention to provide a guidance device for medical devices such as needles that can be quickly and easily manipulated using stereotactic parameters (the angle and depth information) forecasted by a software program product based on the imaging parameters obtained through CT scan/MRI scan.

It is a further object of the present invention to provide an imaging-guided whole-body stereotactic device which is easy to operate and manipulate.

It is further objective of the present invention to provide an imaging-guided whole body stereotactic device that's suitable for intervening different parts of the human body including brain, thorax, abdomen and even limbs.

Embodiments of the present invention broadly discloses an imaging-guided whole body stereotactic device adaptable to align and precisely orient a variety of medical devices/instruments such as differently sized needles, electrodes etc. into a patient's body for carrying out intended medical procedures in a minimally invasive manner. The proposed device is an assembly of multiple parts accompanied by a software program product. The device assembly mainly consists of a base portion, a ring portion, an arc portion, and an instrument guide assembly that are operationally interconnected connected for intended operation.

It is further objective of the present invention to provide angular resolution expansion pack comprising of the arc portion, and the instrument guide for achieving desired base angle and arc angle. The ring portion may be chosen with no offset (in multiples of 2) or an offset of 0.05 degree, 0.1 degree, 0.5 degree, 1 degree 1.5 degree or other values, whereas the instrument guide may be chosen with no offset (in multiples of 2) or with an offset of 0.5 degree, 1 degree 1.5 degree or other values. The chosen ring portion and the instrument guide are assembled on the base portion and the arc portion to achieve higher angular accuracy based on the stereotactic parameters derived from the software.

These and other features, advantages and different embodiments of the present invention will become apparent from the detailed description below, in light of the accompanying drawings.

### BRIEF DESCRIPTION OF DRAWINGS

Other features and advantages of the invention will become clear from the following description and from the figures of the attached drawings, in which:

FIGS. 1A-1B illustrates a front perspective view, and a rear perspective view of a stereotactic device, according to one exemplary embodiment of the present invention.

FIG. 2 illustrates an exploded view of the stereotactic device of FIG. 1A.

FIG. 3A-3B illustrates a front perspective view and a rear perspective view of the stereotactic device, according to another exemplary embodiment of the present invention.

FIG. 4 illustrates exemplary angular resolution expansion parts for the stereotactic device of FIG. 1A.

FIG. 5 illustrates a bottom perspective view of the stereotactic device of FIG. 1A with a ring portion, a base portion, and fiducial wells.

FIG. 6 illustrates engagement of the ring portion with an arc portion according to one exemplary embodiment.

FIG. 7 illustrates a cross sectional view of the stereotactic device of FIG. 1A.

FIG. 7A illustrates an enlarged view of a section ‘A’ shown in FIG. 7, wherein section ‘A’ shows a fiducial marker assembly, according to an exemplary embodiment of the present invention.

FIG. 8 illustrates an instrument guide and an instrument guide cover of the stereotactic device of FIG. 1A in an exploded view.

FIG. 9 illustrates a top view of an exemplary configuration of the instrument guide and instrument guide cover assembled together.

FIG. 10 illustrates the stereotactic device of FIG. 1A in use with the device placed on a head (shown on a right hand side) and an abdomen area (shown on a left hand side) of a human body for precisely guiding a needle or similar instrument to a targeted location in the body.

FIG. 11 illustrates a front perspective view of the stereotactic device, according to yet another exemplary embodiment of the present invention.

FIG. 12 illustrates an exploded view of the stereotactic device of FIG. 11.

FIG. 13 illustrates an instrument stopper configured for use on the stereotactic device of FIG. 11.

FIG. 14 illustrates a cross sectional view of the stereotactic device of FIG. 11.

FIG. 15 illustrates different stereotactic parameters based on which the device can be manipulated or adjusted to guide the instrument to the targeted location of the body for carrying out procedures.

FIG. 16 shows derivation of the stereotactic parameters from spherical coordinate system for a desired target location, wherein the spherical coordinate system has origin O acting as a master center of the stereotactic device.

FIG. 17 illustrates a top perspective view of a base portion of the stereotactic device of FIG. 11.

FIG. 18 illustrates a bottom perspective view of the base portion of the stereotactic device of FIG. 11.

FIG. 19 illustrates a top perspective view of a ring portion of the stereotactic device of FIG. 11.

FIG. 20 illustrates a bottom perspective view of the ring portion of the stereotactic device of FIG. 11.

FIG. 21 illustrates a front perspective view of the ring portion with an arc portion mounted thereon, in accordance with the embodiment of FIG. 11.

FIG. 22 illustrates a back view of the arc portion of FIG. 21.

FIG. 23 illustrates a front and rear perspective views of the instrument guide of the stereotactic device of FIG. 11.

FIG. 24 illustrates a front and rear perspective views of the instrument guide cover of the stereotactic device of FIG. 11.

FIG. 25 illustrates 0 degree (E), 0.5 degree, 1 degree, 1.5 degree angular resolution expansion pack for the stereotactic device of FIG. 11 comprising the ring portion, and the instrument guide that can be selectively mounted over the base portion and the arc portion respectively.

FIG. 26 illustrates 0 degree (E), 0.5 degree, 1 degree, 1.5 degree angular resolution expansion pack comprising the instrument guide for the stereotactic device of FIG. 11.

FIG. 27 illustrates the stereotactic device of FIG. 11 assembled with a unique combination of the angular resolution expansion parts (the ring portion, and the instrument guide) for achieving the desired base angle and arc angle.

FIG. 28 illustrates the concept used to localize the stereotactic device of FIG. 11 using fiducial markers.

FIG. 29A illustrates an exemplary embodiment of a fiducial marker assembly in an exploded view used for Computed Tomography (CT) that encapsulates a metallic fiducial marker.

FIG. 29B illustrates an exemplary embodiment of the fiducial marker assembly in an exploded view for Magnetic Resonance Imaging (MRI) that encapsulates a non-metallic fiducial marker.

FIG. 30 illustrates the fiducial marker assembly in an assembled state with a top fiducial cap engaged on top of a bottom fiducial cap encapsulating the fiducial marker.

FIGS. 31A and 31B illustrates other exemplary embodiment for the fiducial marker assembly with just the bottom fiducial cap.

FIG. 32 illustrates the fiducial marker assembly of FIG. 31B in use.

FIG. 33 illustrates an exemplary user interface of the software that shows different parameters for assembling the stereotactic device obtained from a stereotactic device related program product after the user inputs the parameters/data obtained from CT and/or MRI scans.

FIG. 34 illustrates a method for obtaining stereotactic parameters and use of the stereotactic device of the present invention for performing medical procedure on a patient.

FIG. 35 illustrates the stereotactic device of the present invention in use on the head of a child.

FIG. 36 illustrates the stereotactic device of the present invention in use on the abdomen of an adult.

FIGS. 37A-37B illustrates respectively a front perspective view, and a rear perspective view of a stereotactic device, according to yet another exemplary embodiment of the present invention.

FIGS. 38A-38B illustrates a top perspective view, and a bottom perspective view of a base portion of the stereotactic device of FIG. 37A respectively.

FIGS. 39A-39B illustrates a front perspective view, and a rear perspective view of a ring portion of the stereotactic device of FIG. 37A respectively.

FIGS. 40A-40B illustrates a front perspective view, and a rear perspective view of an arc portion of the stereotactic device of FIG. 37A respectively.

FIGS. 41A-41B illustrates a front perspective view and a rear perspective view of an instrument guide of the stereotactic device of FIG. 37A respectively.

FIGS. 42A-42B illustrates a front perspective view and a rear perspective view of an instrument guide cover of the stereotactic device of FIG. 37A respectively according to one embodiment.

FIGS. 43A-43B illustrates a front perspective view and a rear perspective view of an instrument guide cover of the stereotactic device of FIG. 37A respectively according to another embodiment.

### DETAILED DESCRIPTION

Some embodiments, illustrating its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise. Although any methods, and systems similar or equivalent to those described herein can be used in the practice or testing of embodiments, the preferred methods, and systems are now described. The disclosed embodiments are merely exemplary.

References to “one embodiment”, “an embodiment”, “another embodiment”, “an example”, “another example”, “alternative embodiment”, “some embodiment”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in an embodiment” does not necessarily refer to the same embodiment.

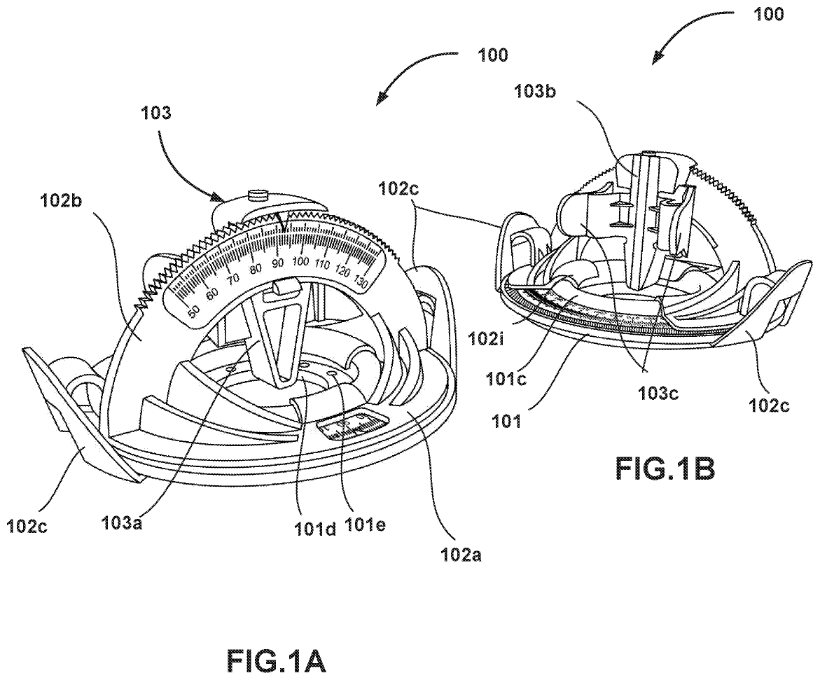

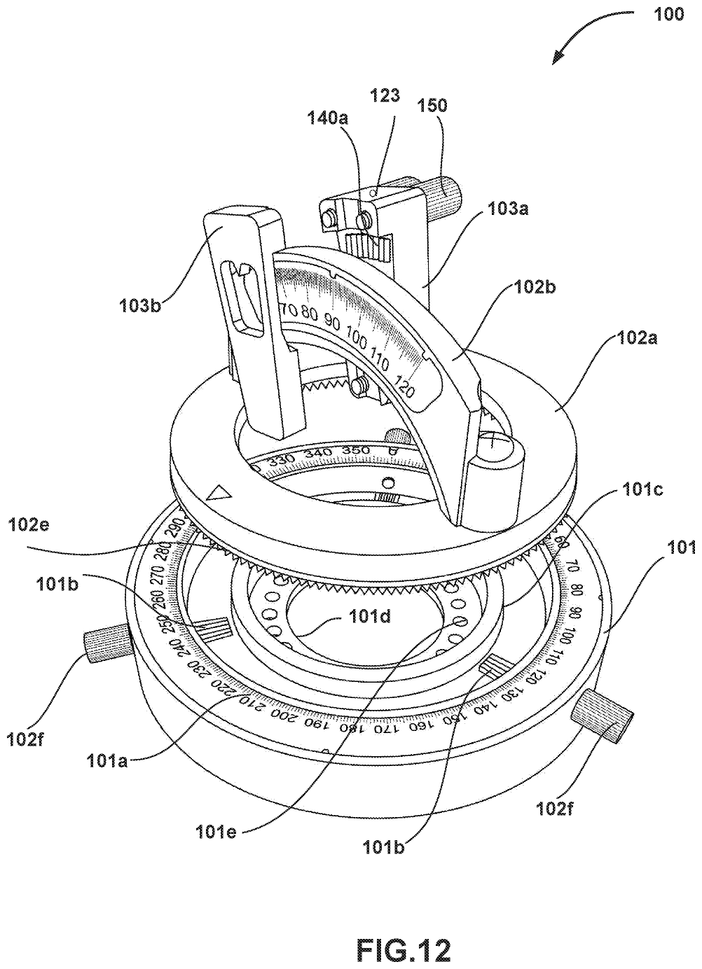

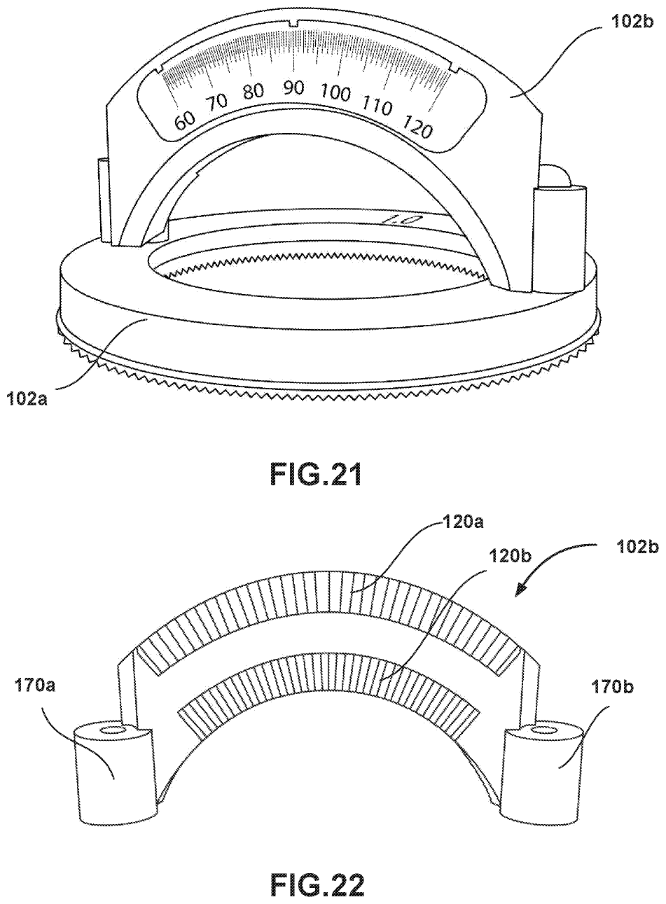

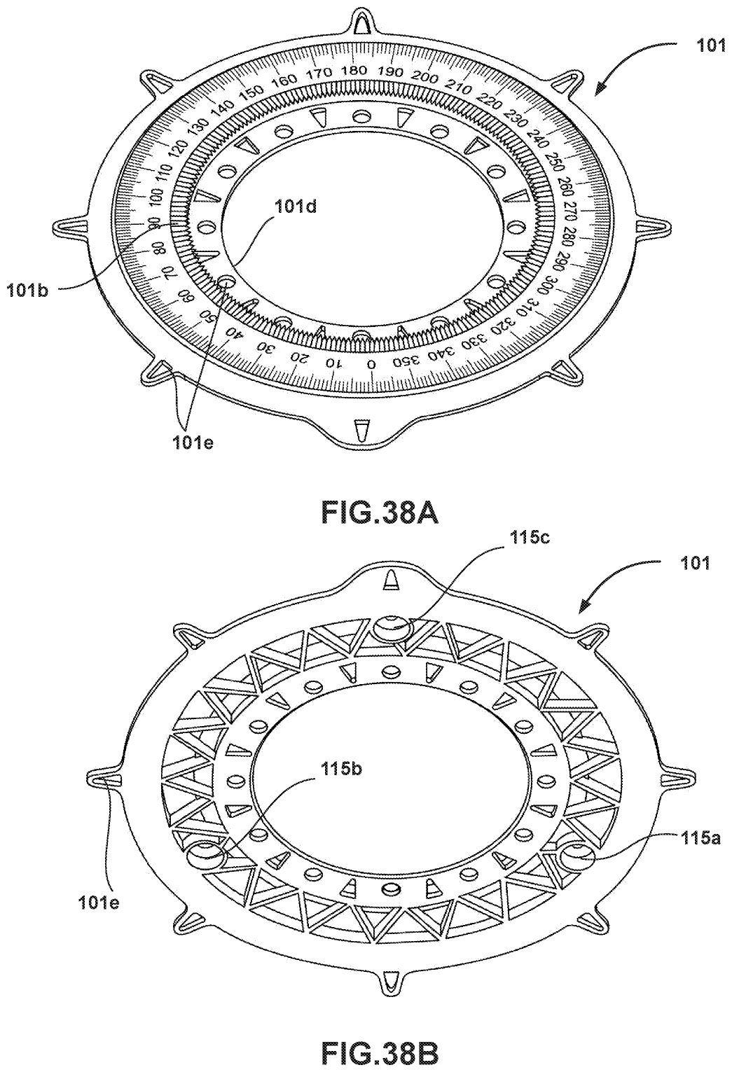

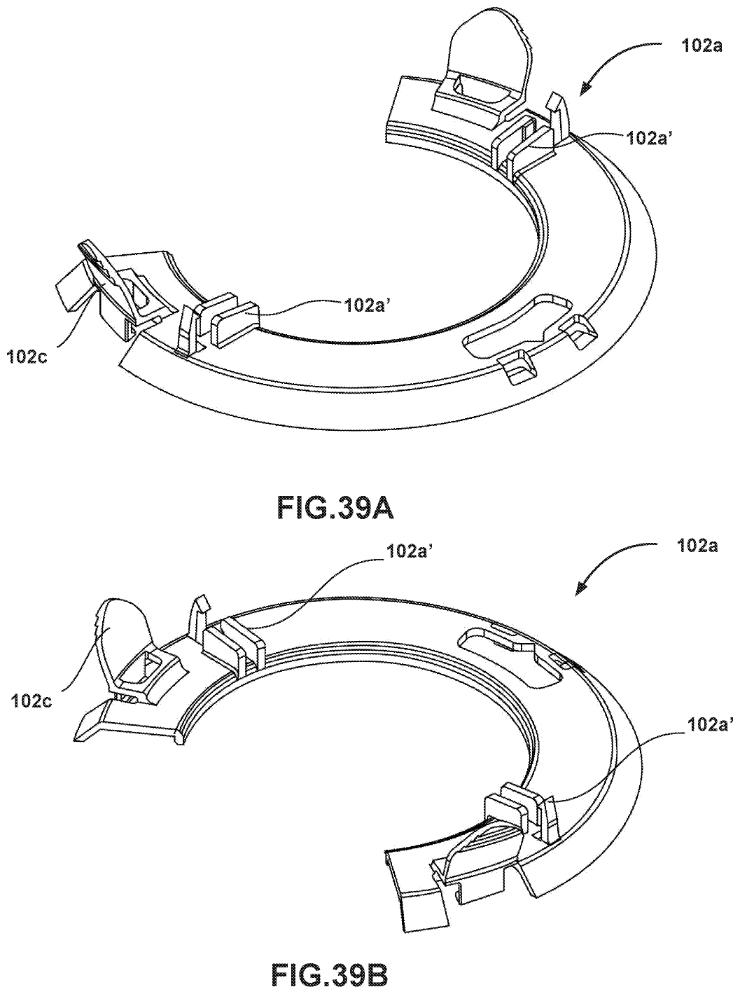

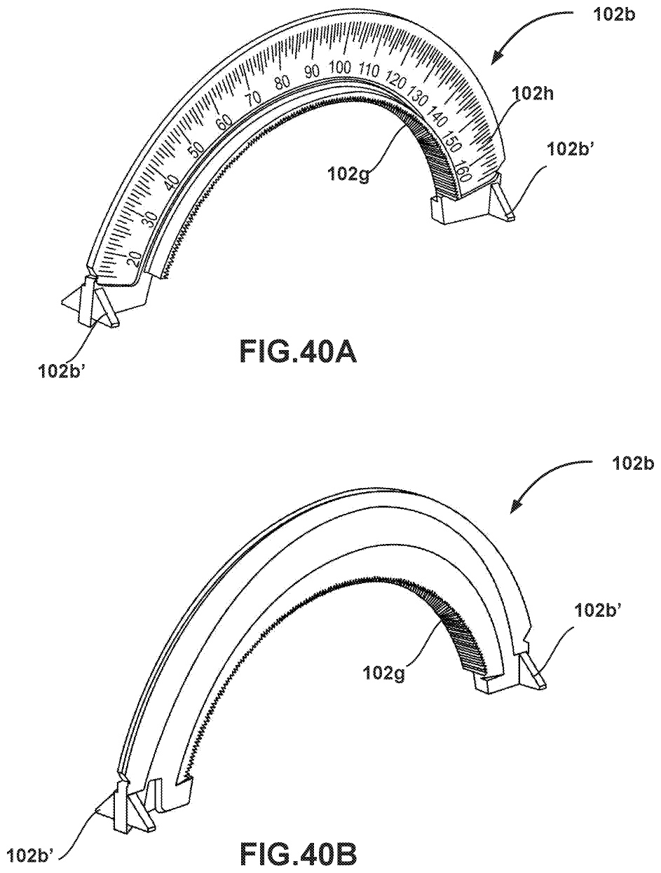

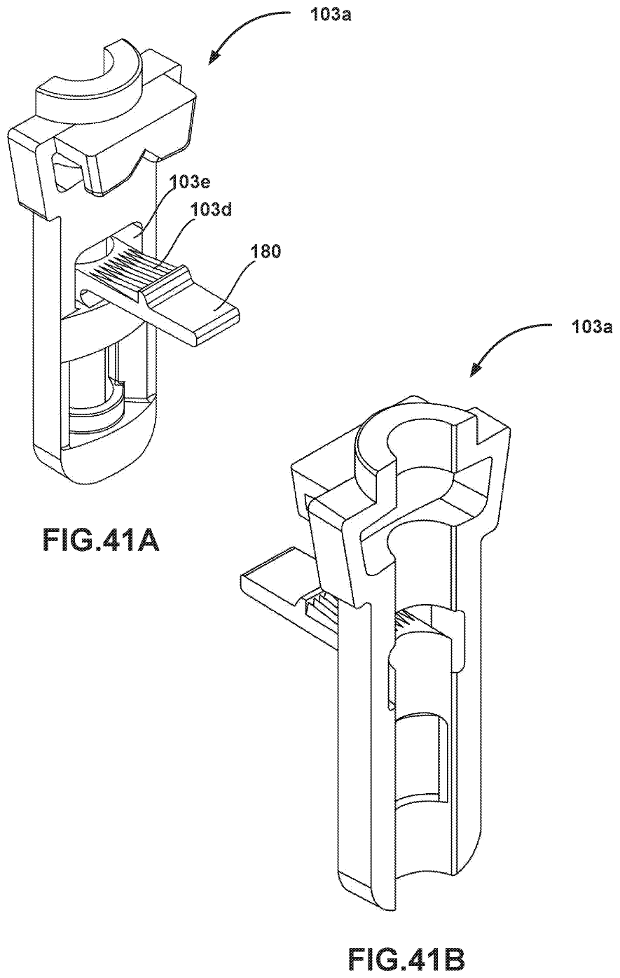

The proposed imaging-guided stereotactic device is an assembly consisting of multiple parts and accompanied by a software program product. The stereotactic device is designed to precisely guide a given instrument towards a target point within a patient's body for carrying out various medical procedures. The various features and embodiments of the present invention are better explained in conjunction with FIGS. 1-43.

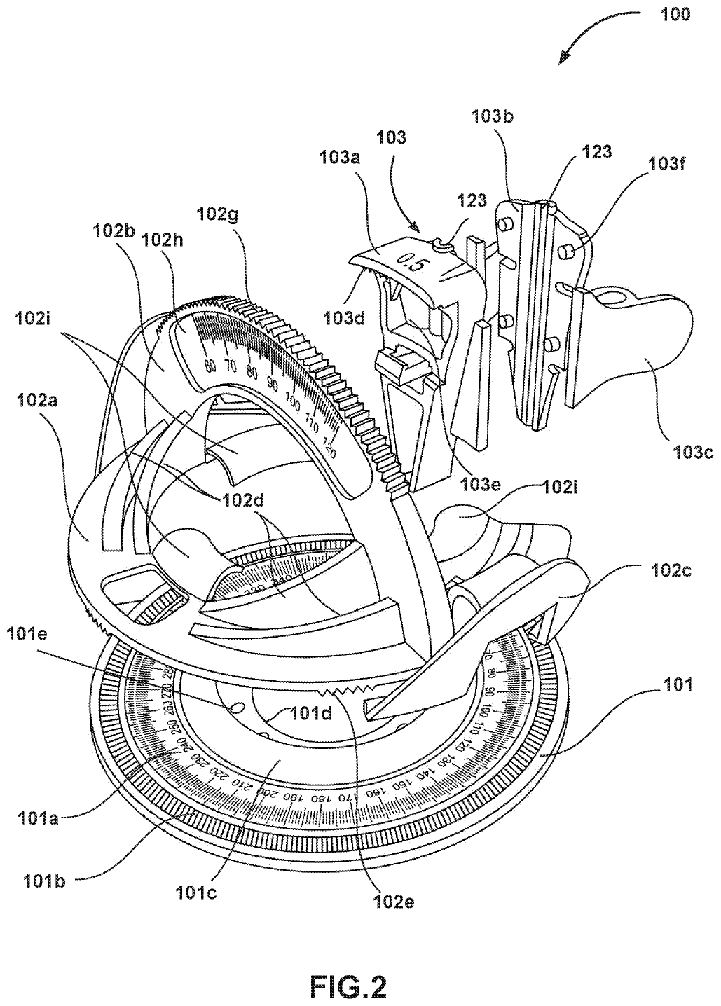

Referring to FIGS. 1A-1B, FIG. 2, the stereotactic device 100 according to one embodiment of the present invention is shown. This embodiment of the stereotactic device 100 is preferably suitable for whole body intervention including but not limited to the brain, and abdomen. As shown, the stereotactic device 100 comprises a base portion 101, a ring portion 102a, an arc portion 102b, and an instrument guide assembly 103 that includes an instrument guide 103a, and an instrument guide cover 103b.

The base portion 101, the ring portion 102a, the arc portion 102b, the instrument guide assembly 103 are designed to be more conveniently and nearly instantaneously disengaged during the procedure once the instrument 90 is placed at the desired location/target using the device 100. This is done by radially disengaging (relative to the base portion 101) the ring portion 102a, the arc portion 102b (or sub-assembly thereof) and the instrument guide assembly 103 from the instrument 90, once the instrument 90 is placed at the desired location/target. During the use of the device 100 for guiding the instrument 90 to a target point, the patient may be asked to hold his/her breath. Once the instrument 90 reaches the target point, the instrument guide assembly 103 is disengaged followed by the disengagement of the ring portion and arc portion sub-assembly, if so desired, all within the same breath hold of the patient, and the patient is then instructed to resume normal breathing. This allows the instrument 90 to move along with the patient's breathing motion without any constraints. Clinically, this minimizes the risk of damage to the internal organs by the instrument 90. For the purpose of this application, the term “instrument” refer to differently sized needles, electrodes and other medical devices that may be used for carrying out medical procedures (such as for example tumor localizations, tumor biopsies) on different parts of the patient's body.

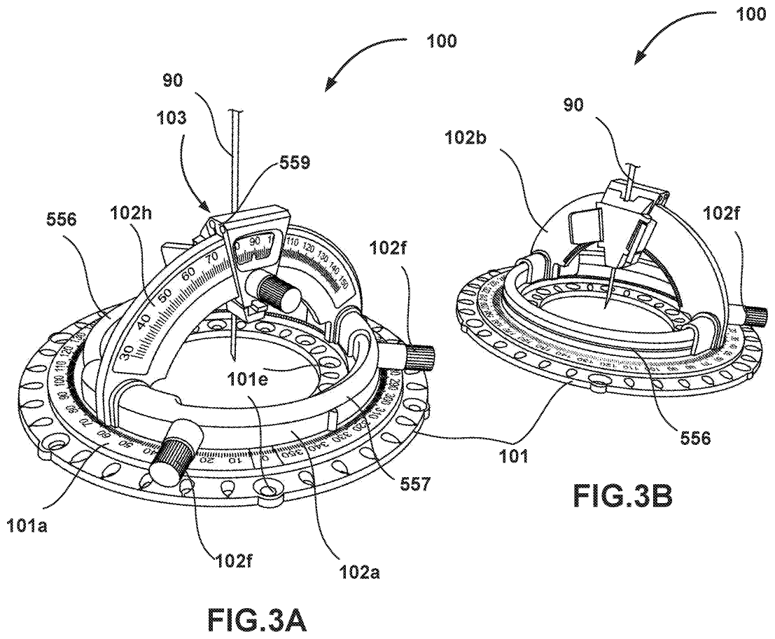

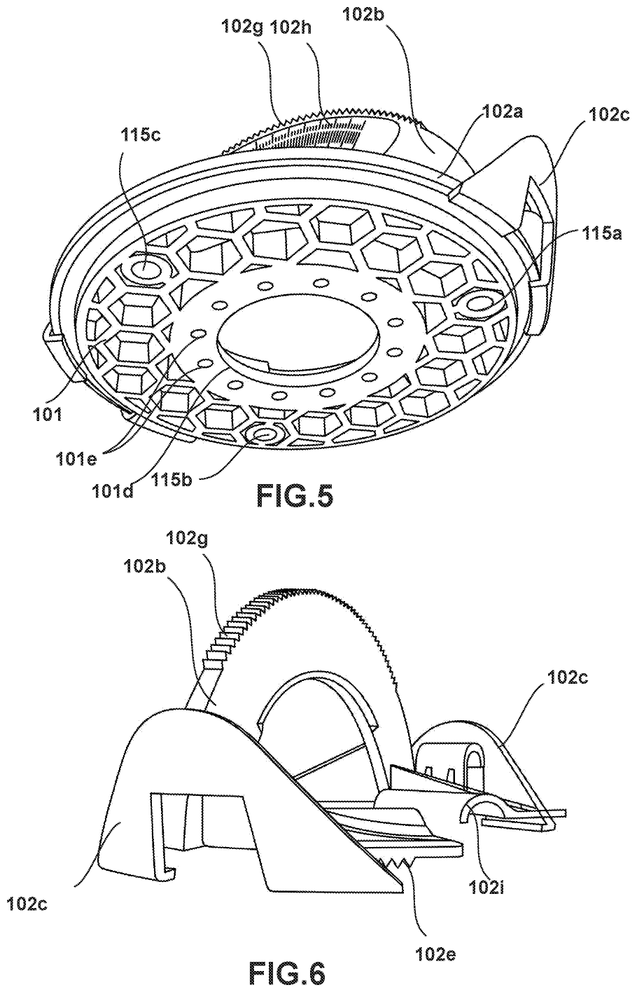

Referring to FIG. 2 along with FIGS. 5-9, the base portion 101 is preferably made circular in shape with reference markings 101a representative of base angles ranging from 0-360 degrees. The base portion 101 further includes angularly separated serrations 101b. The base portion 101 further includes an elevated ring or rail 101c configured to circularly run near the inner edge 101d of the base portion 101. The base portion 101 additionally includes one or more holes/orifices 101e located in a region between the inner edge 101d and the elevated ring 101c or located along the inner circumference of the base portion 101. These holes 101e may be used for affixation of the base portion 101 on the body. The device 100 (particularly the base potion 101) can be screwed and/or sutured on the body at any desired location through these holes 101e. Although in the embodiment shown in FIG. 2 the holes/orifices 101e are seen present along the inner circumference of the base portion 101. It should be understood that these holes/orifices 101e may also be provided on the outer circumference of the base portion 101 as shown in FIG. 3A.

The ring portion 102a is preferably made semicircular or partially circular representing a portion of a circle with one or more pairs of provisions 102d extending upward from the body of the ring portion 102a to fixedly or removably mount the arc portion 102b relative to the ring portion 102a to form a ring portion-arc portion sub-assembly. In some embodiment, the arc portion 102b may be integrally formed on the ring portion 102a as one unitary part rather than the arc portion 102b being removably attached to the ring portion 102a. The bottom surface of the ring portion 102a comprises one or more sets of spaced apart angularly separated serrations 102e. The relative affixation or removable attachment and rotational positioning of the ring portion 102a with respect to the base portion 101 is achieved by means of serrations 101b and 102e configured on the base portion 101 and the ring portion 102a, respectively. These serrations 101b and 102e are configured on the base portion 101 and the ring portion 102a respectively to ensure the ring portion 102a remains in place immobile relative to the base portion 101 and doesn't move when the medical procedure is being carried out or during respiration or other activities. Although the embodiments disclosed herein show or describe the presence of serrations 101b and 102e on the ring portion 102a and the base portion 101 for their relative attachment. It should be understood that many other similar arrangements that would removably attach the ring portion 102a over the base portion 101 to ensure that these two pieces remain immobile may be employed. For example, in some exemplary embodiment, the bottom surface of the ring portion 102a may include holes or slots (not seen) arranged in a continuous or non-continuous manner at the outer periphery instead of serrations 102e and upwardly protruding pins (not seen) arranged on the base portion 101 in a continuous or non-continuous fashion in place of the serrations 101b to enable engagement between the ring portion 102a and the base portion 101. Further, in one embodiment, as shown in FIG. 2, the ring portion 102a may further be assembled to the base portion 101 by means of clips 102c. The clips 102c may be made as an integral part of the ring portion 102a or may be removably engaged with the ring portion 102a for attachment of the ring portion 102a to the base portion 101. In another embodiment as shown in FIGS. 3A and 3B, the ring portion 102a may be assembled with the base portion 101 by means of screws 102f. The ring portion 102a further includes inwardly curved engaging members 102i that help in engagement of the ring portion 102a with the base portion 101 via the rail 101c.

The arc portion 102b includes reference markings 102h representative of arc angles ranging from one angular value to another angular value for example from 60-120 degrees, 30-150 degrees, 50-130 degrees, and so on depending upon various applications. The arc portion 102b further includes angularly separated serrations 102g configured at preferably, but not limited to, its top curved portion.

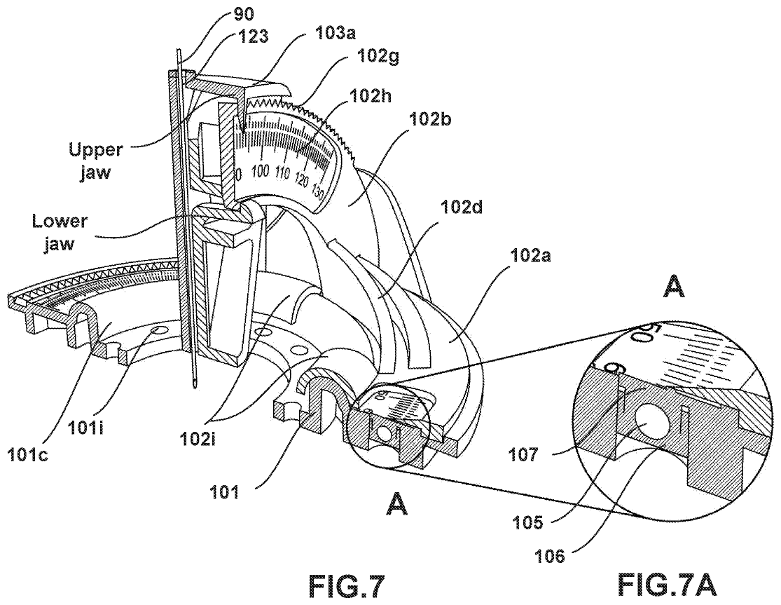

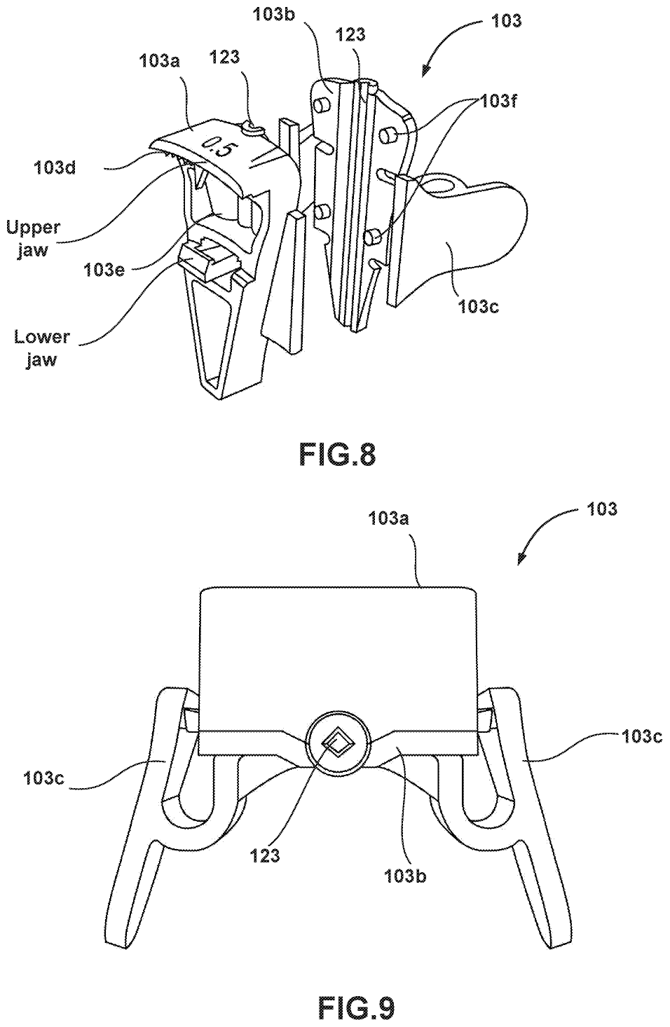

The instrument guide assembly 103 of the device 100 includes the instrument guide 103a and instrument guide cover 103b. The instrument guide 103a is configured to engage or disengage to the arc portion 102b. The exemplary instrument guide 103a shown in FIG. 2 includes a mouth 103e with an upper jaw and a lower jaw (as shown in FIGS. 7 and 8). The upper and lower jaw together with angularly separated serrations 103d configured at bottom surface of the upper jaw helps the instrument guide 103a to mount or dismount to and from the arc portion 102b. The serrations 102g of the arc portion 102b engages with the serrations 103d of the instrument guide 103a to facilitate relative affixation. In one other embodiment as shown in FIG. 3A, the instrument guide 103a may be assembled over the arc portion 102b by means of living hinges 559. The instrument guide assembly 103 also includes a conduit 123 configured to guide an instrument (such as a needle) towards a target point or location in the body. Different configurations of the instrument guide 103a may be made to accommodate different instruments of varying cross-sectional shapes and sizes by varying the cross-sectional shape and size of the conduit 123.

The instrument guide assembly 103 further includes the instrument guide cover 103b that may removably or fixedly attach to the instrument guide 103a. The instrument guide cover 103b may snap fit with the instrument guide 103a via one or more protrusions 103f (seen in FIGS. 2 and 8), which may also serve as locators, or optionally, the instrument guide cover 103b may engage with the instrument guide 103a using some suitable fasteners such as screws. The instrument guide cover 103b may further comprise clips 103c facilitate quick engagement and disengagement of the instrument guide cover 103b to and from the instrument guide 103a.

In the embodiment described herein and as shown illustrated in FIGS. 2 and 8, the instrument guide 103a may embody only a portion of the conduit 123 and may require the instrument guide cover 103b to be coupled to complete the conduit 123 for usage. In simple terms, the conduit 123 is partially embodied in each of the instrument guide 103a and the instrument guide cover 103b, and when assembled together, the conduit 123 is fully operational for guiding the instrument therethrough. Thus, in this configuration, the instrument guide cover 103b instead of performing the function of localizing the instrument guide 103a on the arc portion 102b serves to form the conduit 123. If preferred by the user/surgeon, the conduit 123 Fas partially embodied in instrument guide 103a may still be used to guide the instrument just by itself, without the need for assembly with the other half of the partially embodied conduit 123 in the instrument guide cover 103b.

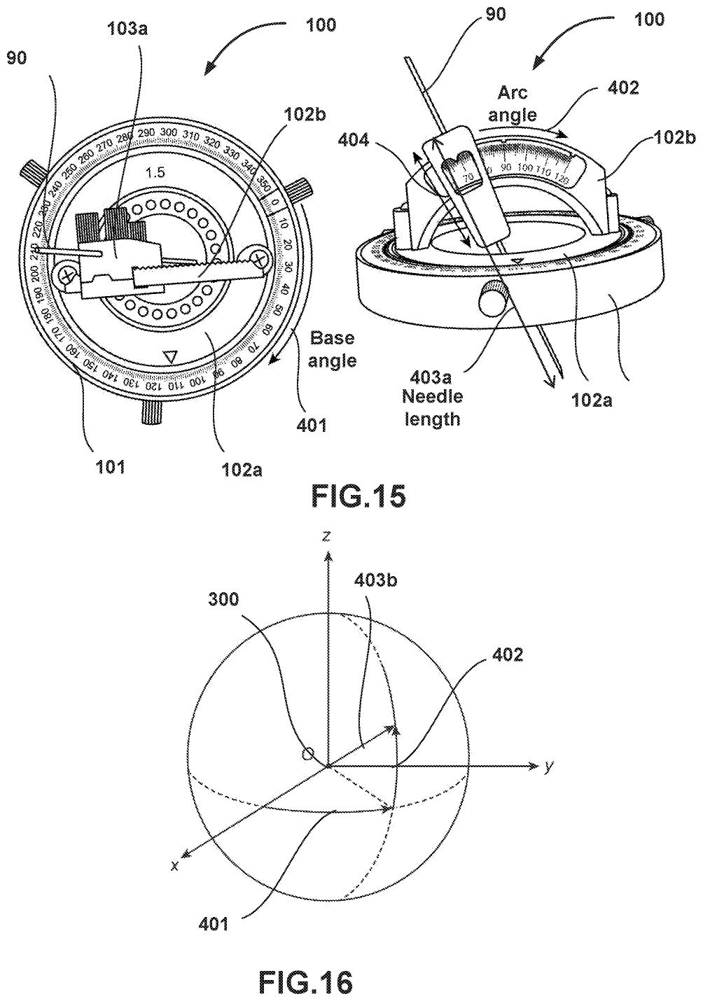

The serrations present on different mating parts of the device 100 described above ensure that the parts are affixed relative to one another by constraining angular/rotational motion about a master center 300 of the device 100 (FIG. 15). Further, as shown in FIGS. 3A and 3B, the device 100 may be constructed without the use of any serrations. As shown in FIGS. 3A and 3B, the base portion 101 may comprise a rail 556, and the ring portion 102a may comprise a saddle 557. The saddle 557 may engage with the rail 556. Further, the screws 102f may be used to facilitate this engagement. The base portion 101 will have angular positions or angles 101a marked on it. Likewise, the arc portion 102b will have arc angles 102h marked on it in order to allow the user of the device 100 to adjust/manipulate the base and arc angle for the instrument being guided.

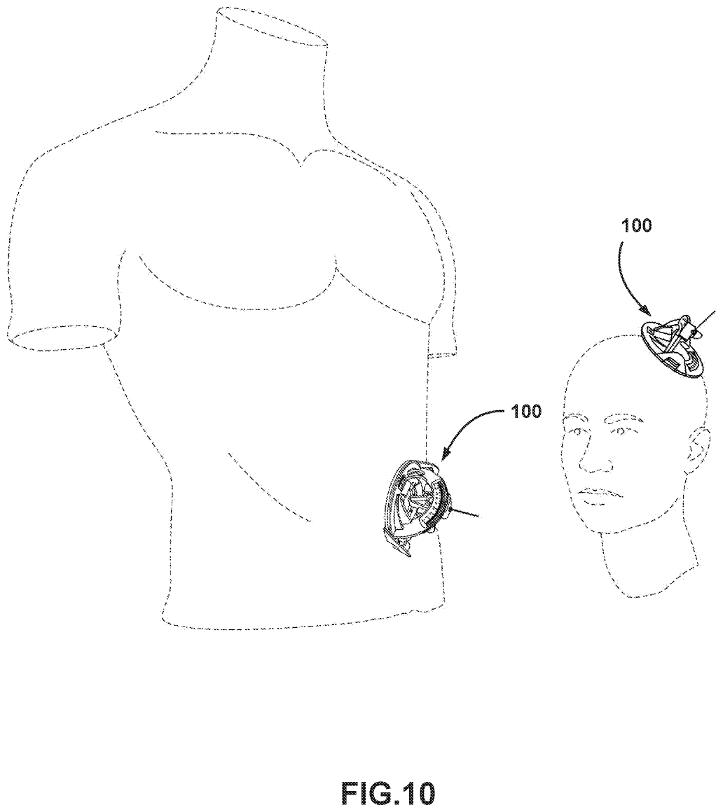

FIG. 10 illustrates the stereotactic device of FIG. 1A in use with the device 100 placed on a head and an abdomen area of a human body for precisely guiding a needle or similar instrument to a targeted location within the brain region and the abdomen region.

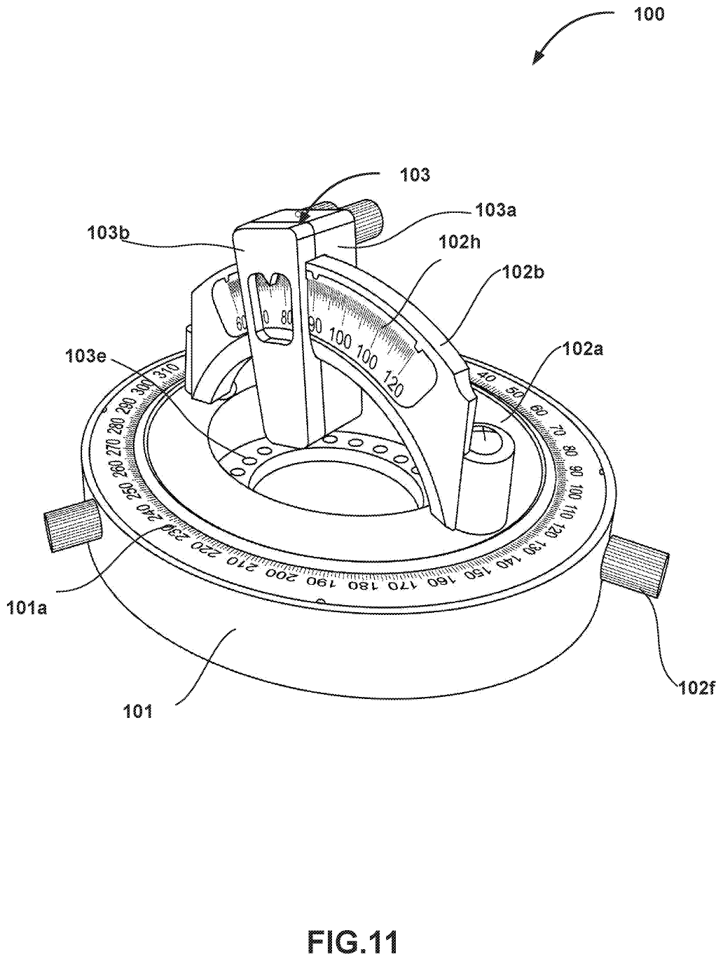

Referring to FIGS. 11-14 and FIGS. 17-24, another embodiment of the stereotactic device 100 of the present invention is shown. The elements and functions of this embodiment shown in FIGS. 11-14 and FIGS. 17-24 is similar to the elements and functions described hereinabove with regard to FIGS. 1A-1B, FIG. 2, FIGS. 3A-3B and FIGS. 5-9 with some exclusive elements or mechanical changes. Accordingly, the detailed description provided hereinabove with respect to FIGS. 1A-1B, FIG. 2, FIGS. 3A-3B and FIGS. 5-9 applies to like numbered elements of FIGS. 11-14 and FIGS. 17-24 and a detailed description of their function is omitted here for conciseness.

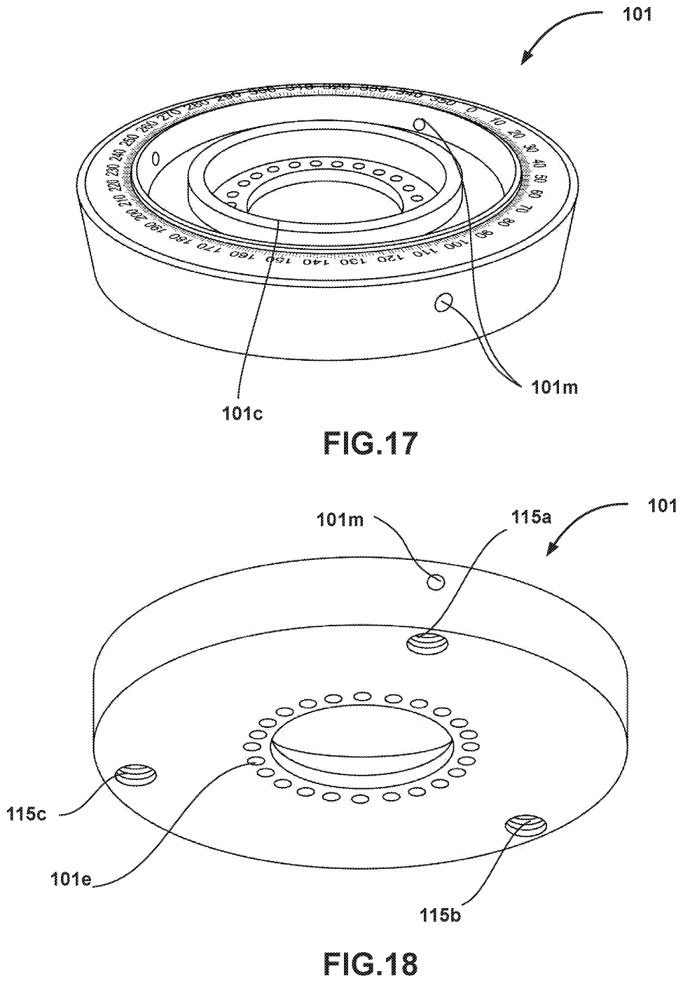

As described hereinabove, the device 100 consists of the base portion 101, the ring portion 102a, the arc portion 102b, the instrument guide 103a and the instrument guide cover 103b. The base portion 101 in this embodiment is similar to the base portion discussed above with exception in terms of the presence of the serrations 101b in the base portion which in this embodiment is non-continuous as seen in FIG. 11. Further, in this embodiment, the base portion 101 also includes screws or thumb screws 102f for facilitating the engagement of the ring portion 102a with the base portion 101. The base portion 101 includes one or more slots 101m (FIG. 17) located at its surrounding wall. The slots 101m are provided on the base portion 101 for the screws 102f to pass through and engage to the ring portion 102a during assembly.

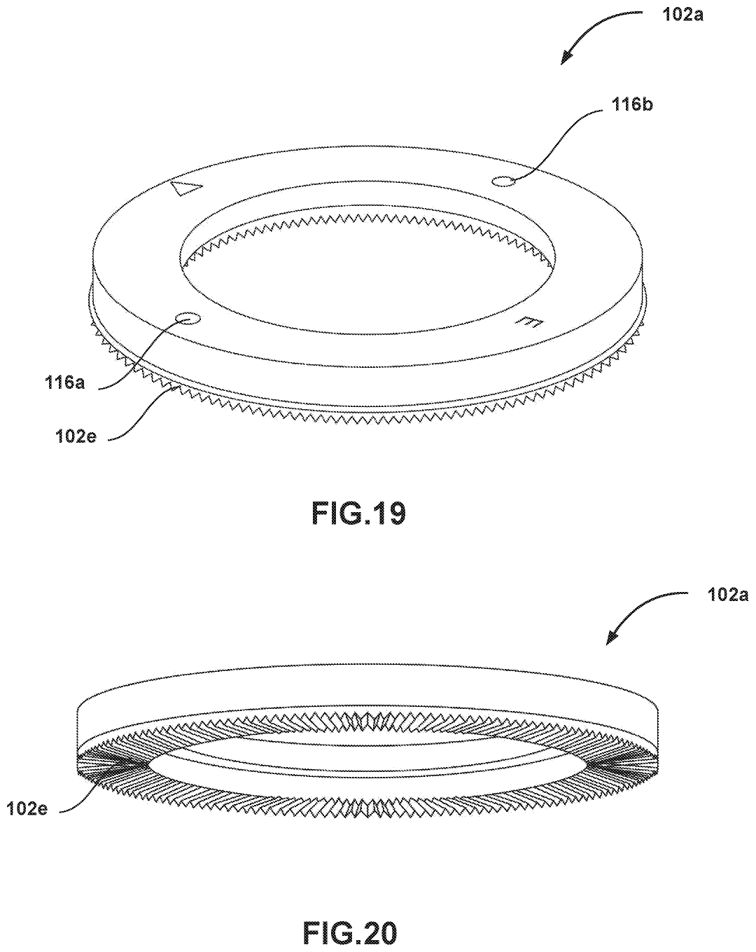

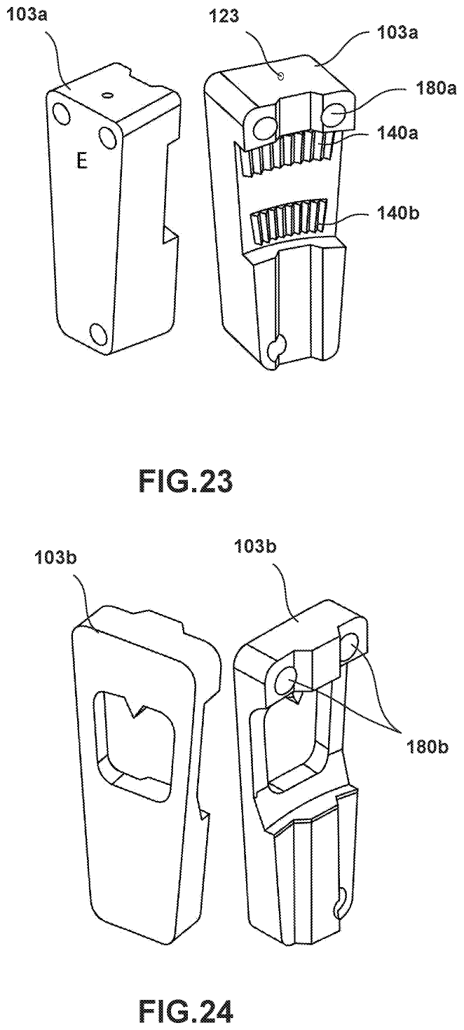

The ring portion 102a in this embodiment is essentially circular compared to being semicircular/partially circular in the embodiments discussed hereinabove. In contrast to the non-continuous serrations 102e, in this embodiment, the serrations 102e are present in continuous fashion at bottom of the ring portion 102a as seen in FIG. 20. The top of the ring portion 102a further includes a pair of slots 116a, 116b (FIG. 19) for facilitating attachment of the ring portion 102a with the arc portion 102b using screws/other assembly means. The arc portion 102b is similar to the arc portion 102b discussed hereinabove in relation to prior presented embodiments except having a pair of angularly separated serrations 120a, 120b (FIG. 22) at its vertical/side face. In some embodiments, these serrations 120a, and 120b may be connected together to form a single set of serrations referred to as 120 (not seen). In other words, the pair of serrations 120a, and 120b present at the verticle/side face may be continuously formed without being spaced apart as seen in FIG. 22. These serrations 120a. 120b configured at the vertical/side face of the arc portion 102b helps in engagement of the arc portion 102b with the instrument guide assembly 103 (particularly engagement with the instrument guide 103a). As seen in FIG. 22, the two ends of the arc portion 102b include provisions 170a, 170b for receiving screws that engages the arc portion 102b with the ring portion 102a. In contrast to the instrument guide 103a discussed hereinabove in other embodiments, in this embodiment, the instrument guide 103a comprises a pair of angularly separated serrations 140a, 140b (FIG. 23) that engages with the serrations 120a, 120b of the arc portion 102b during assembled configuration. Further, in this embodiment, the conduit 123 for the passage of the instrument 90 is located within the instrument guide 103a in contrast to the partial presence of conduit in each of the instrument guide 103a, and instrument guide cover 103b. Thus, in this embodiment, the instrument guide cover 103b performs the function of localizing the instrument guide 103a on the arc portion 102b. The instrument guide cover 103b is constructed to complement to the structure of the instrument guide 103a. The instrument guide 103a comprises slots 180a, and the instrument guide cover 103b comprises slots 180b. The instrument guide 103a and the instrument guide cover 103b are coupled together using screws that pass through the slots 180a, 180b.

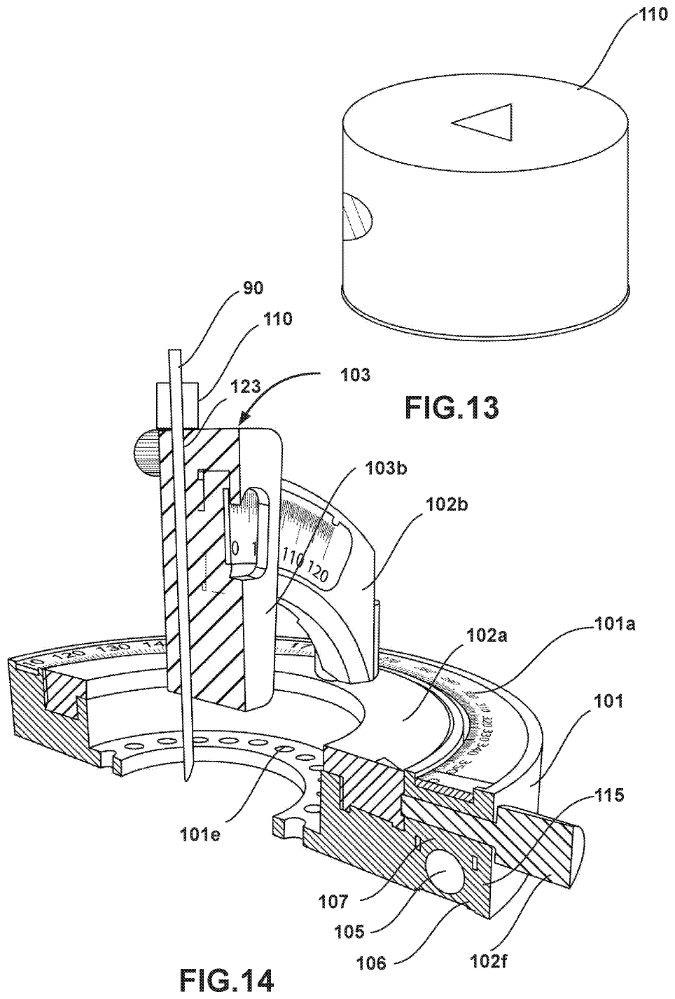

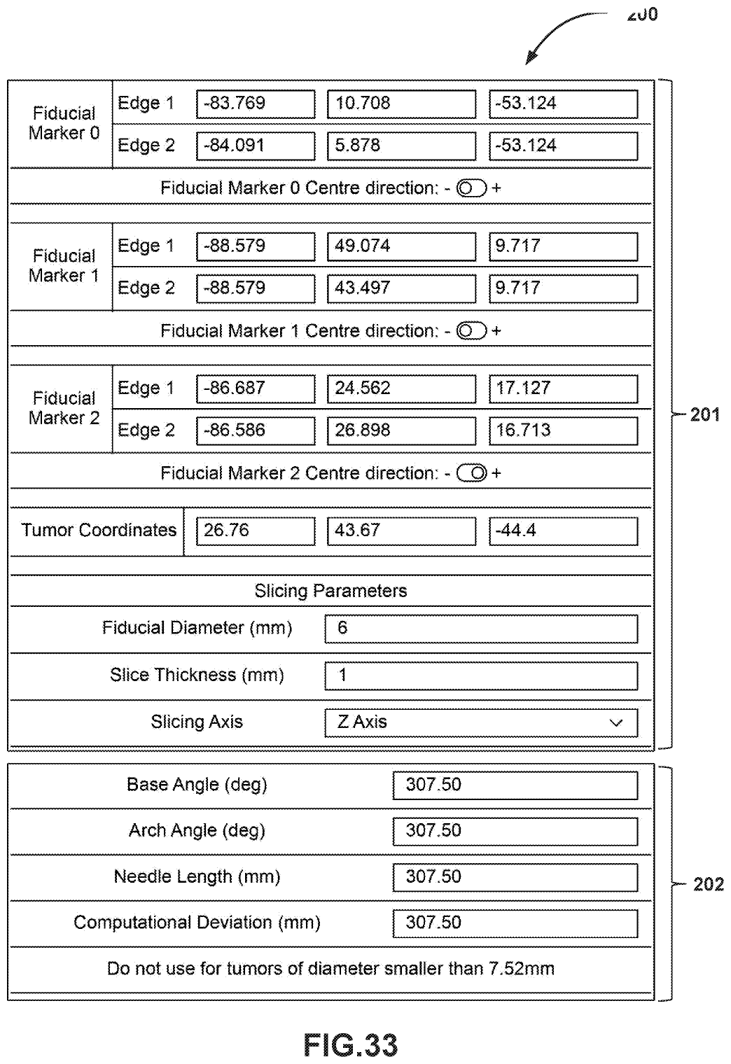

Further according to the embodiment, a user (such as a surgeon) can precisely align the instrument 90 towards a target point/location and set the depth to which an instrument 90 needs to be delivered within a patient by utilising accessory parts such as an instrument stopper 110 (see in FIGS. 13 and 14) using the proposed device 100. The instrument stopper 110 is used to set a fixed length for the instrument 90 allowing the instrument 90 to be passed through the device 100 to no more distance than the distance calculated by the software for the instrument to reach a target point. The manipulation of the device 100 to precisely align and insert the instrument 90 in the body is supported by a software program product accompanying the device 100. The software program product is installable in various user devices such as for example computer, tablet, and phone. When executed, the software program product provides user interfaces such as an interface 200 shown in FIG. 33. The software program product performs the function of calculating the stereotactic parameters (a base angle 401, an arc angle 402 and a depth of insertion or needle length 403a as seen in FIGS. 15 and 16) required by the device 100 to accurately hit a target point within the body. The interface 200 as shown in FIG. 33 includes an input section 201 and an output section 202. The user is prompted to fill in inputs at the input section 201 to assist in localization of the device 100. The inputs given by the user may include coordinates in the form of any coordinate system (for example Cartesian coordinate system) related to the desired target and fiducial markers, imaging modality parameters including slicing parameters, angular resolution desired to be used, fiducial marker types, etc. In an example, these input details are obtainable from the CT scan/MRI scan etc. Once the user inputs or fills in the input section 201, the software program product then processes the inputted information using appropriate software routines/subroutines, logics and algorithm to generate and display the stereotactic parameters (base angle, arc angle, needle length) and other information such as targeting accuracy, type of the ring portion 102a and instrument guide 103a to be used for desired angular resolution, etc. at the output section 202. Once the user has received the two rotational parameters (base angle 401 and arc angle 402) and a translational parameter (instrument length 403a) from accompanying software program product, the user can set the base angle 401 and the arc angle 402 on the device 100 and the instrument length 403a on the instrument 90 with/without the aid of an instrument stopper 110 and perform the procedure.

Referring to FIG. 15 illustrates three geometrical parameters and the manner in which they can be set on the device 100 for guiding the instrument to a desired location in the body. The relative position of the ring portion 102a with respect to the base portion 101 represents the base angle 401 and the relative position of the instrument guide 103a with respect to the arc portion 102b represent the arc angle 402. Utilising the instrument stopper 110 in conjunction with a vernier caliper or other linear distance measuring instruments, the instrument length 403a can be set precisely on the instrument 90, as shown in FIG. 14.

The three geometrical parameters describe a spherical coordinate system with the origin being at the master center 300 of the device 100. The base angle 401 can be considered analogous to yaw angle, the arc angle 402 analogous to roll angle, and the instrument length 403a can be considered analogous to the linear distance value 403b of the spherical coordinate system, with the addition of a device length component 404, as shown in FIGS. 15 and 16. The device length component 404 is the length of the instrument 90 from the master center 300 to the entry point of the conduit 123.

This allows the device to be able to access any point in a volumetric space determined by the range and resolution of base angle and arc angle that can be set on the device 100. For the purpose of this application, any reference to geometrical parameters and stereotactic parameters relate to the arc angle, base angle and the needle length.

The presence of serrations discussed hereinabove with respect to embodiments may be configured in the form of gear teeth, flutes, etc. distributed radially about a central point such as the master center 300. The shape and size of the serrations and the angular separation between are determined by the manufacturability. These serrations may be present or may not be present depending upon availability of other substitute means of constraining angular orientations of the base portion, arc portions, and the instrument guide with respect to each other. In some embodiment, mechanical fasteners and/or mechatronic systems that may use stepper motors may be used to provide the measured angular movement instead of using serrations.

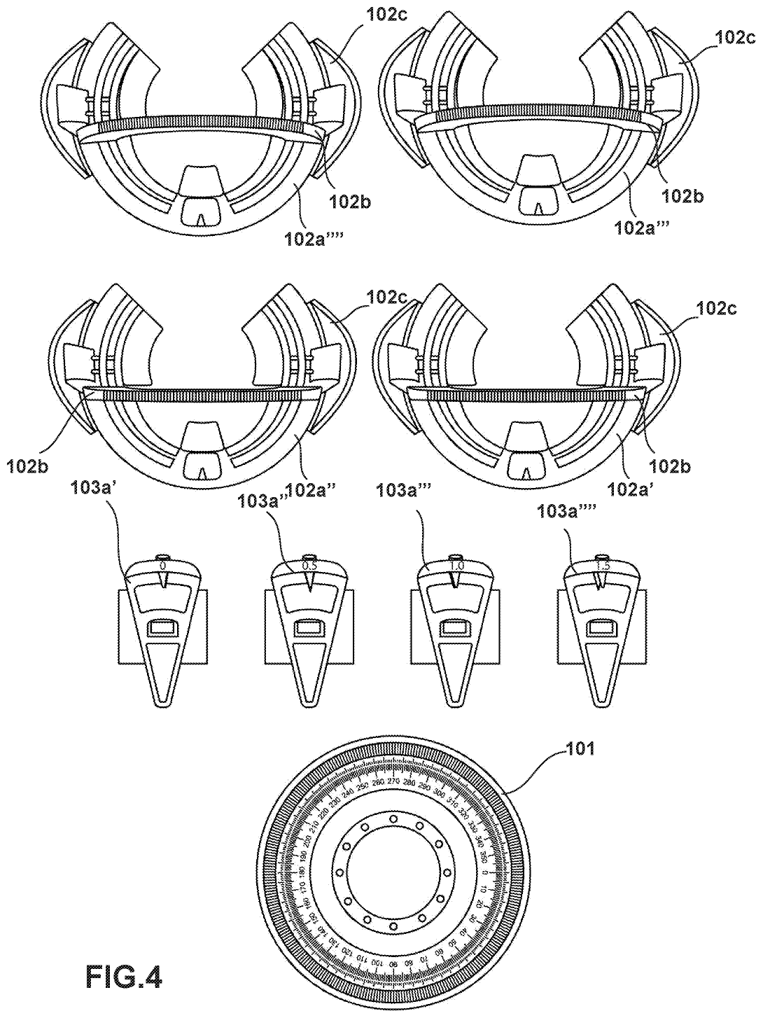

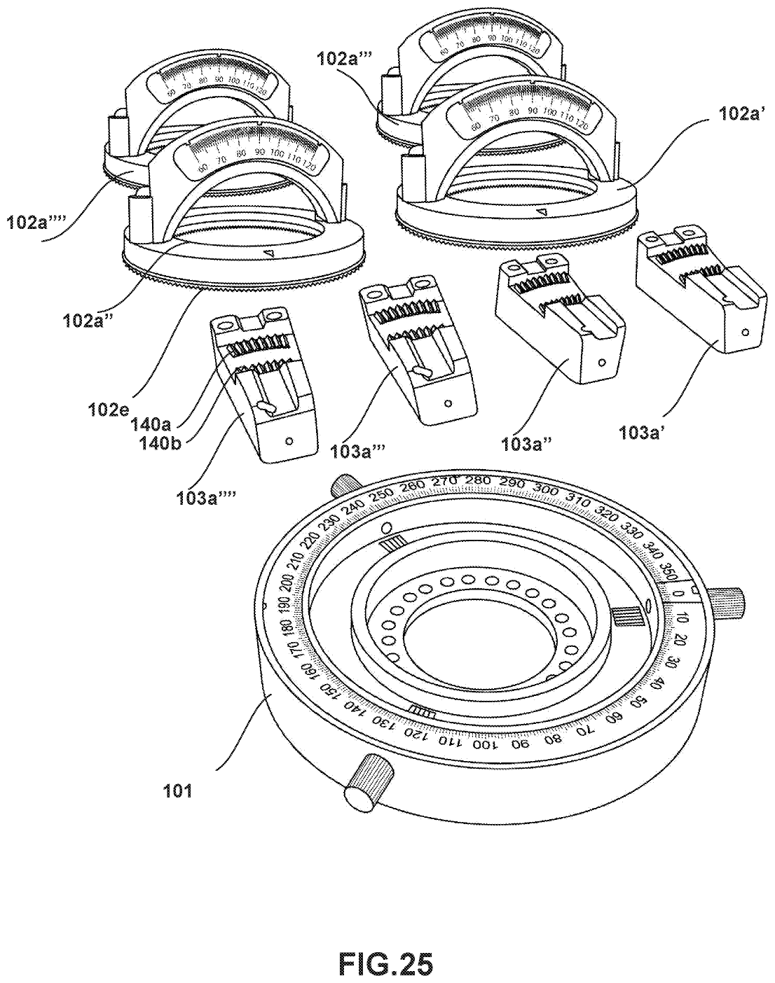

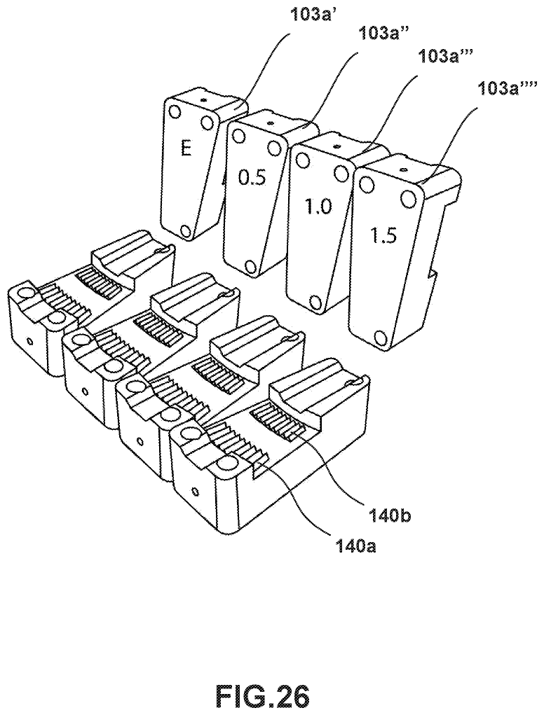

Further, angular resolution provided in the device 100 may be fixed and restrictive to adjustment of the arc angle, base angle in multiples of 2 due to manufacturing constraints. However, practically it is not necessary that the required arc angle, base angles are always in multiples of 2. At times, the small angular values such as 0.05 degree, 0.1 degree, 0.5 degree, 1 degree, 1.5 degrees etc. are important for consideration especially for critical procedures that demand higher accuracy in terms of guidance of the instrument within the body. Keeping this mind, the inventors herein propose additional angular resolution expansion parts as shown in FIG. 4, FIG. 25 and FIG. 26 that can be used as alternatives for instrument guide 103a, and the ring portion 102a and/or the arc portion 102b (including subassembly thereof) in the device 100. FIG. 25 illustrates angular resolution expansion parts required to achieve 0.5 degree angular resolution when the actual angular separation between the serrations is 2 degrees. The angular resolution expansion parts consist of additional configurations of the ring portions (102a′-102a″″) and the instrument guides (103a′-103a″″) that have their serrations 102e, 140a, 140b offset from each other by a fixed angular value. In the illustrated configuration, if the ring portion 102a and instrument guide 103a have their serrations separated by 2 degrees, the base angle and arc angle values can be set at the device 100 without use of any angular resolution expansion parts. The angular values that can be set are limited to multiples of 2. This can be achieved using the angular expansion parts (E) or say (102a′ and 103′). The additional 0.5 degree, 1 degree, 1.5 degree angular resolution expansion parts would consist of one set of the instrument guide and the ring portion (102a″ and 103a″) or (102a′″ and 103a″′) or (102a″″ and 103a″″) for setting an angle that is a multiple of 2 plus/minus 0.5 degree, an angle that is a multiple of 2 plus/minus 1 degree and an angle that is a multiple of 2 plus/minus 1.5 degrees, respectively. Similarly, if an angular resolution of 0.1 degree is required to be achieved the angular resolution expansion parts would include 19 sets with one set for each angle 0.1 degree apart. The instrument guide and ring portion expansion parts may be selectively used i.e. angular expansion part E may be used for the instrument guide (103a′) to achieve the desired arc angle, while angular expansion part 0.5 may be used for the ring portion (102a″) to achieve the desired base angle, so that the combination may provide the highest accuracy for reaching the target lesion.

Besides the use of the additional angular resolution expansion parts (as alternative for instrument guide 103a, and the ring portion 102a and/or the arc portion 102b as shown in FIG. 4, FIG. 25 and FIG. 26 with the device 100 to precisely and accurately guide the instrument 90 at the desired targeted location, the inventor herein also proposes the possibility of having shiftable serrations on the instrument guide 103a, ring portion 102a and/or the arc portion 102b that could be manipulatable by the personnel performing the medical procedure to set any desired angular resolution value. In some embodiment, the instrumentation guide 103a, ring portion 102a, and/or the arc portion 102b may be provided with multiple serrations with the different angular resolution offset values to enable the personnel performing the medical procedure to make use of any desired resolution value sets from the same pieces of the instrument guide 103a, ring portion 102a and/or the arc portion 102b. These embodiments will essentially remove need for having multiple resolution expansion parts.

The parts and components of the device described herein above particularly represent mechanical assembly part of the device 100. Besides the mechanical assembly, the device 100 includes a fiducial assembly which will be described in detail in the description to follow.

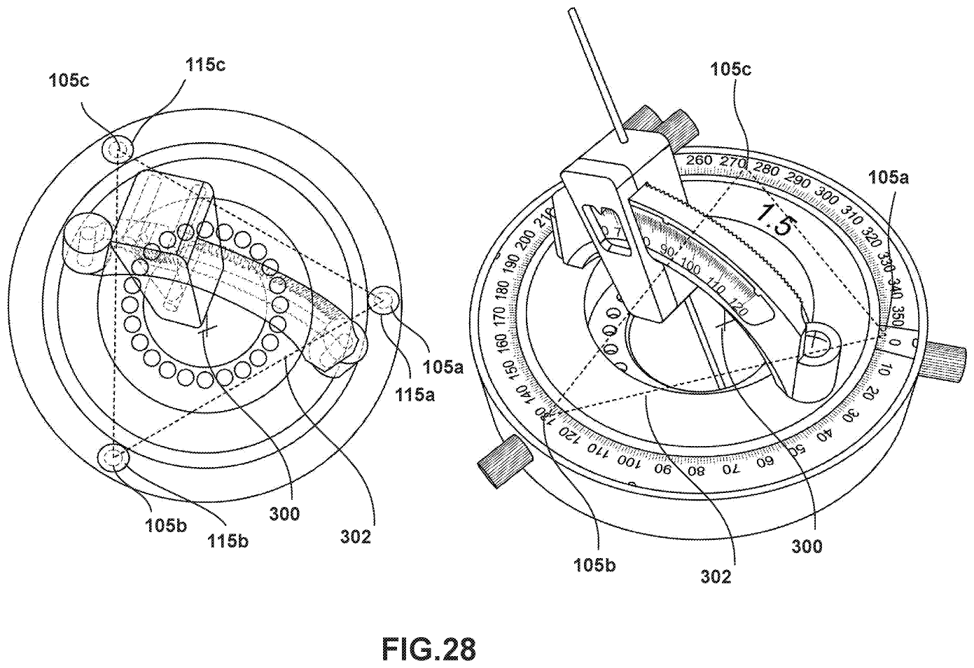

Fiducial Assembly includes fiducial markers 105a-105c as shown in FIG. 28 located within respective fiducial wells 115a-115c configured at bottom surface of the base portion 101 (shown in FIG. 5 and FIG. 18). In one embodiment, the localization of the fiducial markers 105a-105c within the fiducial wells 115a-115c may be achieved using a bottom fiducial cap 106 and/or a top fiducial cap 107, as shown in FIGS. 7A and 14. In another embodiment, the fiducial wells 115a-115c may have their opening located on another surface such as the outermost circumferential surface of the base portion 101. In yet another embodiment, the fiducial markers 105a-105c may be positioned within the fiducial wells 115a-115c that may be located on other parts of the device 100.

As illustrated in FIGS. 14 and 28, the fiducial wells 115 (or 115a-115c) are of a cylindrical form, the fiducial markers 105 (or 105a-105c) are in a spherical form and fiducial caps 106 and 107 have a cylindrical form with features complementing the spherical form of the fiducial markers 105. In some other embodiments, the fiducial wells 115, fiducial markers 105 and fiducial caps 106 and 107 may take any other shape to complement each other in a way that ensures accurate localization of the fiducial markers within the device 100.

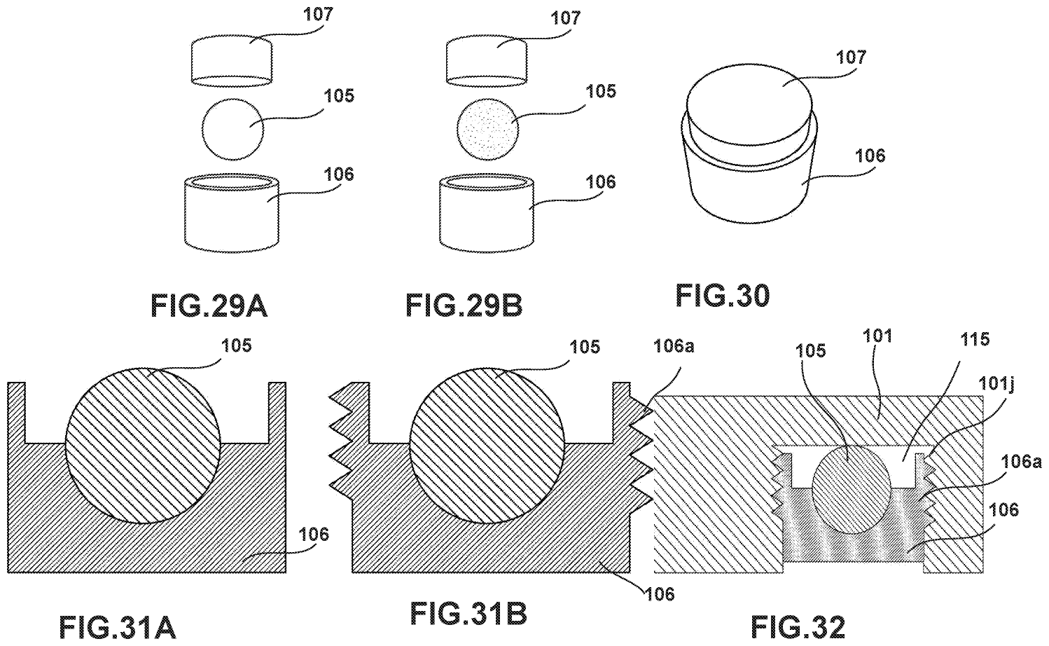

In one embodiment, as shown in FIG. 29A, a fiducial marker assembly in an exploded view used for CT and including a metallic fiducial marker is shown. As seen, the fiducial caps 106 and 107 are designed to encapsulate a solid or metallic fiducial marker 105. In another embodiment, as shown in FIG. 29B, the fiducial marker assembly in an exploded view for MRI and including a non-metallic fiducial marker 105 is shown. The fiducial caps 106 and 107 are designed to encapsulate a non-metallic fiducial marker 105. In yet other embodiments, the fiducial marker may be used in any suitable state of matter and may comprise of multiple materials present in the same or different states of matter e.g. semi-solid, gel, etc. The material encapsulated by the fiducial caps 106 and 107 may vary depending upon the imaging modality that is used for device localization with the respect to a target within the patient.

Although in the preferred embodiments, three fiducial markers 105 are shown assembled in the device 100. In some other embodiments, any other number of the fiducial markers 105 can be assembled onto the base portion 101 or even be configured as a geometrical feature of the base portion 101.

To keep the fiducial marker 105 affixed and localized within the base portion 101, different means such as screws, adhesives, press fit dimensional tolerancing, etc. may be used. In yet another embodiment, the fiducial caps 106, 107 may not be used, and the fiducial marker 105 may be directly attached with the base portion 101 by suitable affixation means such as screws, adhesives, press fit dimensional tolerancing etc.

Referring to FIG. 30, the fiducial marker assembly in an assembled state with the top fiducial cap 107 engaged on top of the bottom fiducial cap 106 encapsulating the fiducial marker 105 is shown. The caps 106, 107 are dimensioned to enable a press fit creating a leak-proof container. This embodiment will allow the use of a liquid, such as oil, fatty lipids, etc., as fiducial markers, which are particularly suited for use with MRI scans. In this embodiment, the fiducial caps 106, 107 will be submerged in the liquid and then press fit encapsulating the liquid in a leak-proof manner. The liquid fiducial marker will bear the shape of the space disposed within the press fit fiducial caps 106 and 107. In some embodiment, the bottom fiducial cap 106 alone may be utilized to localize the fiducial marker 105 within the base member 101. In some embodiment, as illustrated in FIG. 31A, the bottom fiducial cap 106 may have its outermost circumferential surface dimensioned to be able to press fit when inserted into a fiducial well 115. In some other embodiment, as illustrated in FIG. 31B, the bottom fiducial cap 106 may have screw thread like features 106a on its outermost circumferential surface that may complement screw thread-like features 101j configured on the inner circumferential diameter of the fiducial wells 115a-115c as shown in FIG. 32.

In another embodiment, the fiducial markers 105a-105c may be assembled within the fiducial wells 115a-115c by utilizing the bottom fiducial cap 106 and top fiducial cap 107. In an implementation, the bottom fiducial cap 106 may be suitably dimensioned to press fit within the fiducial wells 115a-115c allowing localization of the fiducial markers 105a-105c within the fiducial wells 115a-115c. In another implementation, the fiducial markers 105a-115c may be assembled within the fiducial wells 115a-115c by utilizing the bottom fiducial cap 106 and top fiducial cap 107, for this implementation, the threads may be present on the outermost circumferential surface of the bottom fiducial cap 106 that may then mate with the threads on the inner circumferential surface of the fiducial wells 115a-115c.

The presence of the fiducial assembly as a part of base portion 101 should not be construed as limiting. It should be understood that the fiducial assembly may be formed as a separate independent unit that may be integrated with other parts and any location of the parts of the device 100, such as the instrument guide 103a, the ring portion 102a, the arc portion 102b or a combination thereof. A number of fiducial markers may thus be localized within the device 100 to define an equilateral triangle 302 (as seen in FIG. 28) that may be termed as master plane 302 that define a master center 300 which forms the origin of the spherical coordinate system so formed (shown in FIG. 28). In some other embodiments, other numbers, shapes, and combinations of fiducial markers may be used to define the master plane 302 and master center 300.

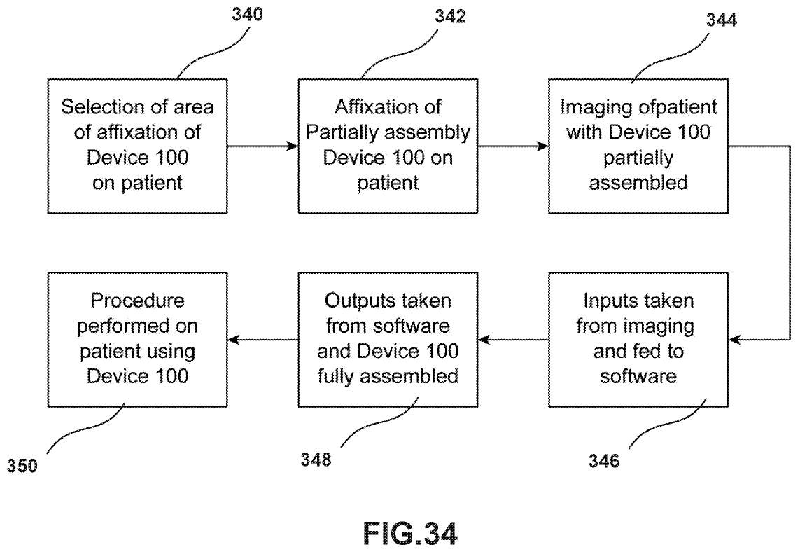

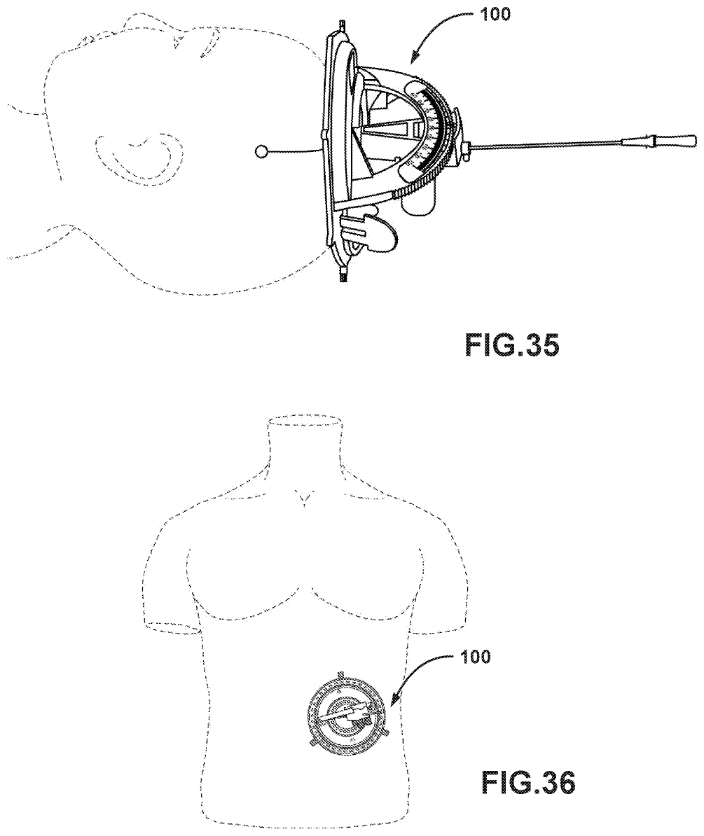

Operation: any of embodiments for the device 100 described hereinabove with respect to FIGS. 11-14 and FIGS. 17-24 or FIGS. 1A-1B, FIG. 2, FIGS. 3A-3B and FIGS. 5-9 may be used for carrying out intended procedure. Referring to FIG. 34 along with FIG. 10 and FIGS. 35-36, the method of operation starts with selection of an area on the patient's body where the device 100 needs to be mounted for carrying out procedure (step 340). Next, the base portion 101 of the device or partially assembled device 100 is affixed to the selected procedural area in proximity to the target point on the patient's body (step 342). The base portion 101 is preferably attached to the body first, since the base portion 101 is CT and/or MRI visible with fiducial markers 105a-105c assembled into it. The means of affixation may vary based on the nature of the location. For example, affixation may be achieved by means of screws passing through the functional orifices/holes 101e present on the base portion 101, if the intended location falls on a bony structure, as is in the case of cranial applications which involves affixation on the skull (see FIGS. 10 and 35). In some other cases, sutures may alternatively be utilized for affixation purpose. Suturability of the device allows for minimizing the trauma to the patient's skull caused due to drilling and the screw holes that may be used to secure the device 100. This has existed as a problem in the prior art teachings thereby proving them inefficient. Securing the device 100 using sutures may be a preferred alternative in many clinical cases, such as in infants where the skull bone may not yet be fully developed. As an example, the device 100 may be used to perform an intervention to take a biopsy sample from the liver—in which case the device 100 will be secured to the abdomen area using sutures. Practically, it is not possible to secure the device 100 using screws in the abdomen area (see FIGS. 10 and 36) due to a lack of sufficient bony/solid structure.

Next, scanning of the patient having the device/base portion 101 attached is done using CT/MRI to obtain different information such as for example, Cartesian coordinates of the target and one or more points on the fiducial markers, preferably either the center or diametrically opposite edges on the circumference. Together with scanning/imaging, viewing/information gathering (e.g. Cartesian coordinates) from the CT/MRI images on a suitable software interface intended to use with the CT/MRI machines is performed (step 344).

This information obtained from the CT/MRI is then fed to the software program product through the input section 201 provided in the user interface 200 (step 346). Once the information from CT/MRI is input into the software program product, the software program product outputs stereotactic parameters available in the output section 202 of the user interface 200 of the software as shown in FIG. 34 (step 348). The stereotactic parameters preferably include a base angle, an arc angle, and a needle length. Next, the user assembles other parts (the ring portion 102a, the arc portion 102b, the instrument guide 103a, and instrument guide cover 103b) of the device 100 and set the relative positions between the base portion 101 and the ring portion 102a (to set intended base angle), and the arc portion 102b and the instrument guide 103a (to set intended arc angle) as per stereotactic parameters obtained from the software program product and the procedure is carried out using the device 100 (step 350). If needed, the user is provided with the resolution expansion parts to achieve a desired level of targeting accuracy as shown and described with respect to FIGS. 4 and 25.

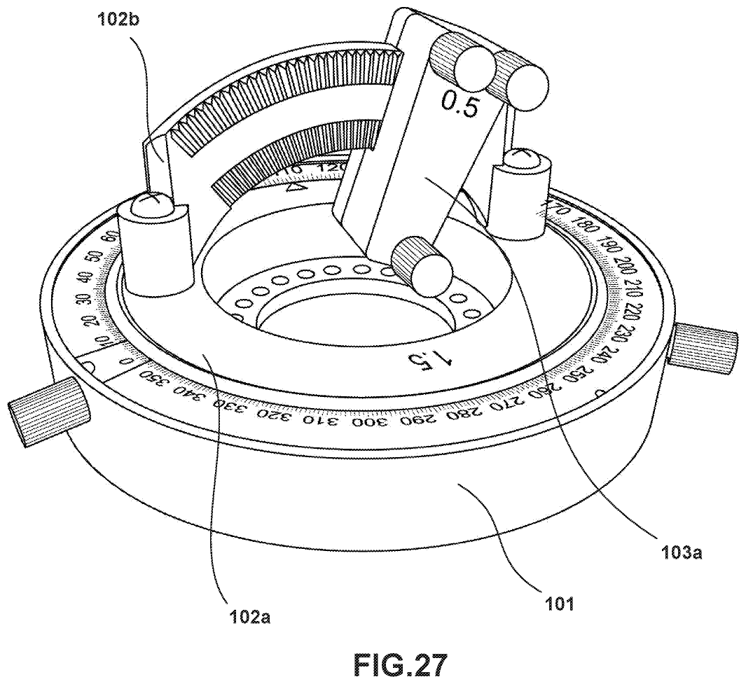

FIG. 27 illustrates the stereotactic device assembled with an exemplary chosen combination of the angular resolution expansion parts (ring portion 102a and instrument guide 103a) for achieving desired base angle and arc angle. With further reference to FIGS. 25 and 26, the ring portion (or ring portion-arc portion subassembly) as seen chosen includes the 1.5 degree angular expansion part (102a″″) whereas the instrument guide as seen chosen includes the 0.5 degree angular resolution expansion part (103a″). The chosen ring portion 102a″″ and the instrument guide 103a″ are assembled on the base portion 101 and the arc portion 102b. This is followed by using an accurate length measuring instrument such as a vernier caliper or a lead screw-based instrument to set the position of the instrument stopper 110 on the instrument 90 (such as needle). The instrument 90 is then pushed through the conduit 123 present on the instrument guide 103a until the instrument stopper 110 comes in contact with the instrument guide 103a. Alternatively, the user may deliver the instrument 90 to the target site without using the instrument stopper 110 while instead utilising an intraoperative imaging technique (e.g. CT/MRI) to see the real time location of the instrument 90 and deem the position of the functional part of the instrument to be sufficiently within or adjacent to the target site.

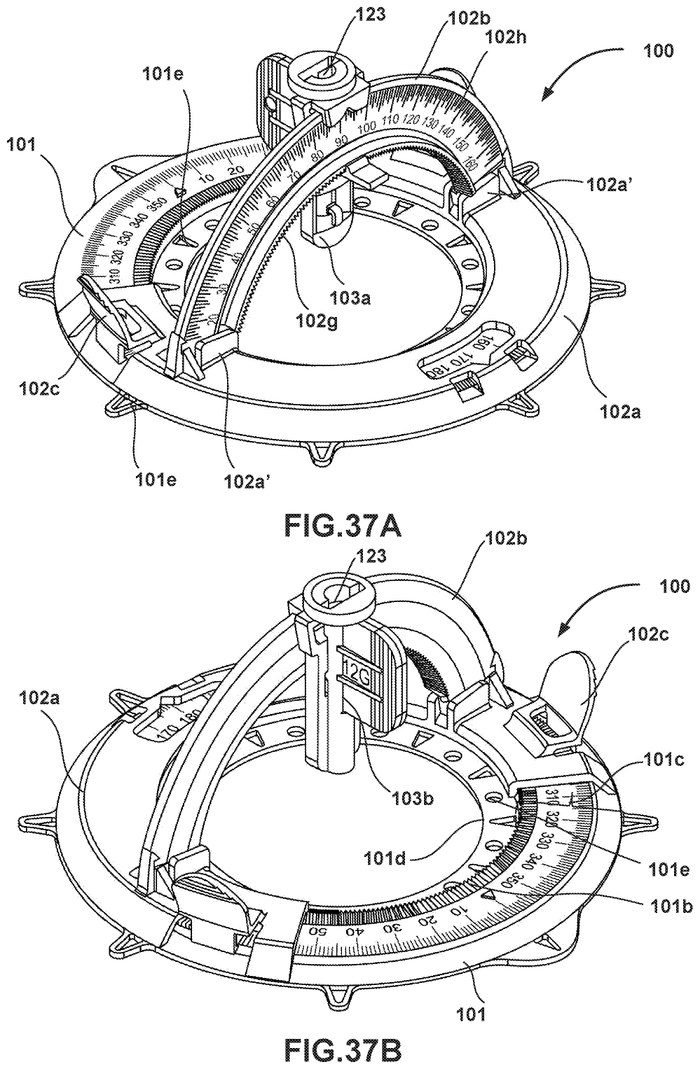

FIGS. 37A-37B to 43A-43B illustrates stereotactic device and associated components thereof, according to yet another exemplary embodiment of the present invention. The elements and functions of this embodiment shown in FIGS. 37A-37B to 43A-43B is similar to the elements and functions described hereinabove with regard to FIGS. 1A-1B, FIG. 2, FIGS. 3A-3B and FIGS. 5-9 with some exclusive elements or mechanical changes. Accordingly, the detailed description provided hereinabove with respect to FIGS. 1A-1B, FIG. 2, FIGS. 3A-3B and FIGS. 5-9 applies to like numbered elements of FIGS. 37A-37B to 43A-43B and a detailed description of their function is omitted here for conciseness.

As described hereinabove, the device 100 consists of the base portion 101, the ring portion 102a, the arc portion 102b, the instrument guide 103a and the instrument guide cover 103b. The base portion 101 in this embodiment is similar to the base portion discussed above with exception in terms of the location of the serrations 101b in the base portion 101. The base portion 101 additionally includes one or more holes/orifices 101e located along the inner circumference of the base portion 101 and also present along the outer circumference of the base portion 101.

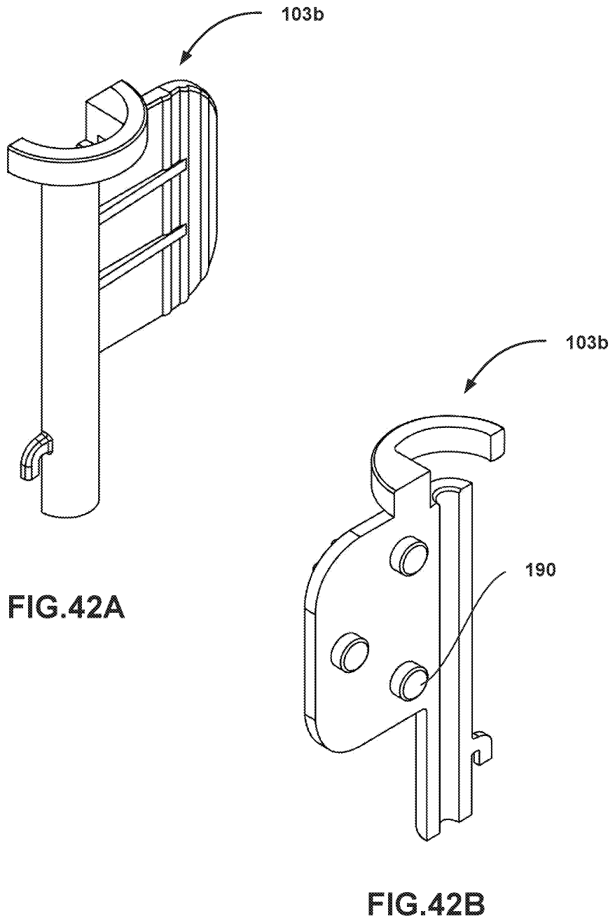

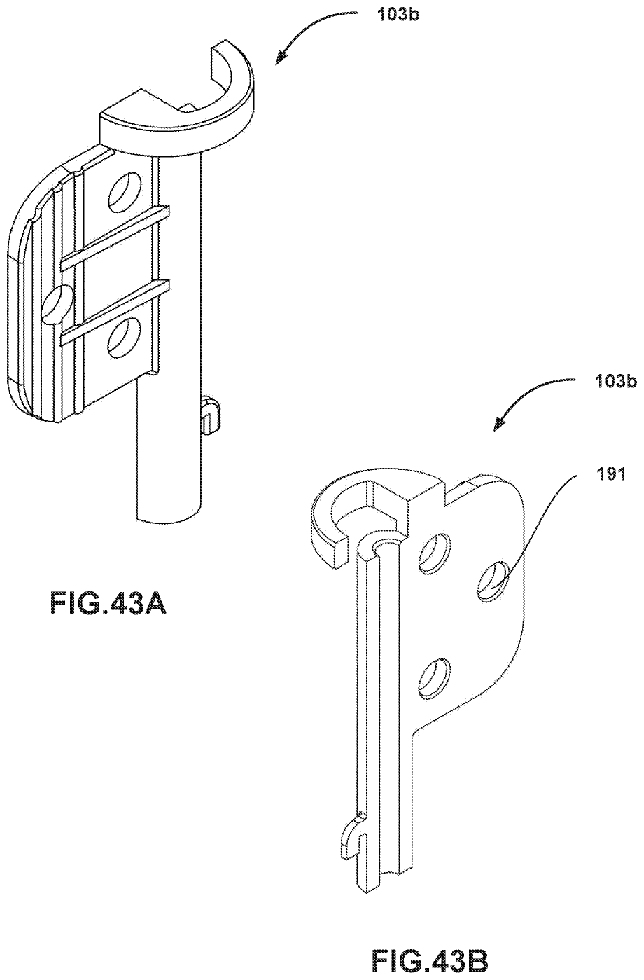

The ring portion 102a in this embodiment is essentially semicircular/partially circular. In this embodiment, the serrations (not seen) are present in continuous fashion at bottom of the ring portion 102a. The top of the ring portion 102a further includes a pair of mounting members 102a′ for facilitating attachment of the ring portion 102a with the arc portion 102b. Particularly, the attachment is snap-fit mechanism. The ring portion 102a may also include clips 102c that may be made as an integral part of the ring portion 102a or may be removably engaged with the ring portion 102a for attachment of the ring portion 102a to the base portion 101 or for ease of removal of the ring portion 102a from the base portion 101 during disassembly. The arc portion 102b is similar to the arc portion 102b discussed hereinabove in relation to prior presented embodiments except having separated serrations 102g configured at preferably, but not limited to, its bottom curved portion. Additionally, provisions 102b′ at the two ends of the arc portion 102b are present to help in engagement of the arc portion 102b with the mounting members 102a′ configured on the ring portion 102a. In contrast to the instrument guide 103a discussed hereinabove in other embodiments, in this embodiment, the instrument guide 103a includes a mouth 103e with a tongue 180 having angularly separated serrations 103d that helps the instrument guide 103a to mount or dismount to and from the arc portion 102b (via serrations 102g). Further, in this embodiment, the conduit 123 for the passage of the instrument is located partially in each of the instrument guide 103a, and instrument guide cover 103b. Unlike the instrument guide cover designs disclosed in the other embodiments, in this embodiment, the instrument guide cover 103b is configured in two pieces (seen in FIGS. 42B and 43B). Each of these opposing pieces are configured to mate with each other to form a unitary piece. The arrangement (slots and pivots) numbered as 190, 191 helps in engagement and disengagement of two piece instrument guide cover 103b.

Some of preferred embodiments of the present invention are described hereinabove, the device 100 may also be modified to accommodate actuators, electronic systems, etc. to robotically move the parts of the device 100 relative to each other, while controlling/monitoring such movement remotely, as per the required stereotactic parameters. Further, one or more of aforementioned embodiments discussed above may be combined to improve manufacturability, targeting accuracy, user experience, aesthetics, etc. Further, the components/parts of the device 100 may be made using variety of material that's biocompatible and may be made in different dimensions and thus use of material and size of the device 100 should not be construed to be a limiting factor.

The preceding description has been presented with reference to various embodiments. Persons skilled in the art and technology to which this application pertains will appreciate that alterations and changes in the described structures and methods/steps of operation can be practiced without meaningfully departing from the principle, spirit and scope of the present invention.

1. An imaging-guided whole-body stereotactic device (100) adaptable to align and guide a medical instrument (90) towards a target location in a human body during an intended medical procedure, the stereotactic device (100) comprising:
a base portion (101) including a first set of reference markings (101a) representing base angles (401) ranging from 0-360 degrees, and one or more orifices (101e) located along at least one of:
an inner circumference, and an outer circumference thereof for affixation of the base portion (101) on the human body;
a ring portion (102a) removably affixable and rotationally positionable over the base portion (101);
an arc portion (102b) including a second set of reference markings (102h) representing arc angles (402) ranging from one specific angular value to another angular value;
an instrument guide assembly (103) having an instrument guide (103a), and an instrument guide cover (103b) removably or fixedly attachable to the instrument guide (103a), the instrument guide (103a) is configured to engage or disengage to or from the are portion (102b), wherein the instrument guide assembly (103) comprises a conduit (123) configured to guide the medical instrument (90) towards a target location in the human body;
wherein, the base portion (101), the ring portion (102a), the are portion
(102b), and the instrument guide assembly (103) are configured to be at least
conveniently disengaged, and nearly instantaneously disengaged during the intended medical procedure; and
wherein, the device (100) is manipulatable by varying a plurality of stereotactic parameters including at least the base angles (401), the are angles (402), and a depth of insertion of the instrument or instrument length (403a) derived from a software program product in order to hit the desired target location using the device (100).

2. The stereotactic device (100) of claim 1, wherein the ring portion (102a) further comprising a plurality of inwardly curved engaging members (102i) that help in engagement of the ring portion (102a) to an elevated rail or ring (101c) provided on the base portion (101).

3. The stereotactic device (100) of claim 1 further comprising one or more sets of spaced apart angularly separated serrations (102e) at bottom surface of the ring portion (102a) for connecting the ring portion (102a) to one or more sets of angularly separated serrations (101b) provided on the base portion (101) in order to ensure the ring portion (102a) remain immobile in place relative to the base portion (101).

4. (canceled)

5. (canceled)

6. The stereotactic device (100) of claim, 1 wherein the ring portion (102a) further comprising one or more pair of provisions (102d) extending upward from the body of the ring portion (102a) to fixedly or removably mount the are portion (102b) relative to the ring portion (102a) or the ring portion (102a) comprises a pair of slots (116a, 116b) on top for facilitating attachment of the ring portion (102a) with the are portion (102b) using fasteners such as screws.

7. The stereotactic device (100) of claim 1 further comprising a plurality of clips (102c) integrally formed on the ring portion (102) or removably engaged with the ring portion (102a) for attachment of the ring portion (102a) to the base portion (101)

8. (canceled)

9. The stereotactic device (100) of claim 1, wherein the base portion (101) comprising one or more slots (101m) located at its surrounding wall for facilitating one or more thumb screws 102f to pass therethrough to engage the ring portion (102a) to the base portion (101).

10. (canceled)

11. The stereotactic device (100) of claim, 1 wherein the instrument guide (103a) comprises one or more living hinges (559) that helps in mounting or dismounting of the instrument guide (103a) to and from the arc portion (102b).

12. (canceled)

13. The stereotactic device (100) of claim 12, wherein the instrument guide cover (103b) comprises one or more pairs of clips (103c) to facilitate quick engagement and disengagement of the instrument guide cover (103b) to and from the instrument guide (103a).

14. (canceled)

15. (canceled)

16. The stereotactic device (100) of claim 1, wherein the base portion (101) comprises a rail (556), and the ring portion (102a) comprises a saddle (557) that engages with the rail (556).

17. The stereotactic device (100) of claim, 3 wherein the angularly separated serrations (102e)
and the angularly separated serrations (101b) connects the ring portion (102a) on the base portion (101) by constraining angular/rotational motion about a master center (300) of the device (100).

18. The stereotactic device (100) of claim 3, wherein the angularly separated serrations (102e) are non-continuously configured or continuously configured at bottom of the ring portion (102a).

19. The stereotactic device (100) of claim 1, wherein the are portion (102b) comprising a pair of angularly separated serrations (120a, 120b) or angularly separated serrations (120) configured at the vertical/side face to help the are portion (102b) in engagement with the instrument guide assembly (103).

20. The stereotactic device (100) of claim 1, wherein the two opposing ends of the are portion
102b comprises a pair of provisions (170a, 170b) for receiving screws that engages the arc portion (102b) with the ring portion (102a).

21. The stereotactic device (100) of claim 19, wherein the pair of angularly separated serrations (120a, 120b) located on the are portion (102b) engages with a pair of angularly separated serrations (140a, 140b) configured on the instrument guide (103a) during the device assembly.

22. The stereotactic device (100) of claim 1, wherein the software program product receives at least coordinates in the form of a cartesian coordinate system related to the desired target location, one or more fiducial markers, one or more imaging modality parameters including slicing parameters, angular resolution desired to be used, fiducial marker types obtainable from a CT scan/MRI scan in order to calculate the plurality of stereotactic parameters.

23. (canceled)

24. The stereotactic device (100) of claim 1, wherein the plurality of stereotactic parameters further comprising type of the ring portion (102a) and the instrument guide (103a) to be used for a desired angular resolution.

25. (canceled)

26. (canceled)

27. (canceled)

28. The stereotactic device (100) of claim 24, wherein the desired angular resolution is adjustable using angular resolution expansion parts consisting of additional configurations for the ring portions (102a′-102a″″) and the instrument guides (103a′-103a″″) that have their serrations 102e, 140a, 140b offset from each other by a fixed angular value.

29. The stereotactic device (100) of claim 28, wherein the fixed angular value comprises at least 0.05 degree, 0.1 degree, 0.5 degree, 1 degree, and 2 degrees.

30. The stereotactic device (100) of claim 1 further comprising one or more fiducial assembles, each of the fiducial assemblies including a fiducial marker (105a-105c) located within respective a fiducial well (115a-115c) configured at the base portion (101) and enclosed using a bottom fiducial cap (106) and/or a top fiducial cap (107).

31. The stereotactic device (100) of claim 30, wherein the fiducial marker (105a-105c is a solid fiducial marker (105) and/or a non-solid fiducial marker (105) comprising a suitable state of matter so as to be visible distinctly from other parts of the device (100) upon imaging with CT/MRI scan.

32. (canceled)
